(19) United States
(12) Patent Application Publication
(10) Pub. No.: US 2025/0296900 A1
(43) Pub. Date: Sep. 25, 2025

(54) DECOMPOSITION OF POLYOLEFINS

(71) Applicant: OXFORD UNIVERSITY INNOVATION LIMITED, Oxford (GB)

(72) Inventors: Shik Chi Edman TSANG, Oxford (Oxfordshire) (GB); Jianwei ZHENG, Oxford (Oxfordshire) (GB)

(21) Appl. No.: 18/727,730

(22) PCT Filed: Jan. 19, 2023
(86) PCT No.: PCT/GB2023/050088

§ 371 Date: Jul. 10, 2024

(30) Foreign Application Priority Data
Jan. 19, 2022 (GB) 2200663.9

(51) Int. Cl. C07C 4/22; B01J 29/40; B01J 35/61; B01J 35/64; C08J 11/16

(52) U.S. Cl. CPC C07C 4/22; B01J 29/40; B01J 35/615; B01J 35/643; C08J 11/16

(57) ABSTRACT

The present invention relates to a process for the catalytic decomposition of a polyolefin. More particularly, the present invention relates to the aromatic-aided catalytic decomposition of polyolefins under mild conditions using a zeolite.

### CROSS-REFERENCE TO RELATED APPLICATIONS

This application is the National Stage of International Application No. PCT/GB2023/050088, filed Jan. 19, 2023, which claims priority to GB 2200663.9, filed Jan. 19, 2022, which are entirely incorporated herein by reference.

### INTRODUCTION

The present invention relates to a process for the catalytic decomposition of a polyolefin. More particularly, the present invention relates to the aromatic-aided catalytic decomposition of polyolefins under mild conditions using a zeolite.

### BACKGROUND OF THE INVENTION

Synthetic plastics are found in almost every aspect of modern life and finding viable recycling routes is of critical importance. Although efforts have been made to tackle this problem, the ongoing production of plastic waste, which has increased during the global coronavirus pandemic¹⁻³, means that pressure on regular waste management practices has increased.

Mechanical recycling of plastic waste by melting and re-extrusion is one such waste management practice but is often considered as “downcycling” due to the presence of residual catalysts, moisture, and other contaminants, leading to unwanted products⁴. By contrast, chemical catalytic recycling of waste plastic has shown more promise on a workable scale⁵. Zhang et al. transformed waste polyethylene (PE) into long chain alkylbenzenes, a feedstock for detergent manufacture, by coupling exothermic hydrogenolysis with endothermic aromatization using a platinum/alumina catalyst⁶. Tennakoon et al.⁷ mimicked the enzyme-catalysed conversions of biomacromolecules to the selective hydrogenolysis of high-density polyethylene (HDPE) into a narrow distribution of diesel and lubricant-range alkanes. Similarly, attempts at converting thermal plastics into diesel range products by fluid catalytic cracking (FCC) catalysts have also been made^{2,8,9}. Jie et al.¹⁰ adopted the microwave-initiated catalytic deconstruction of plastic waste into hydrogen and carbon nanotubes using Fe-based catalysts.

A different and particularly attractive route for recycling waste plastics involves converting plastics back into monomers and subsequent repolymerisation. This closed loop strategy conforms to many of the standards set by governments, agencies and manufacturers in order to meet sustainability goals. Attempts at obtaining a closed loop strategy using enzymatic processing of polyethylene terephthalate (PET) and polyester^{11,12} as well as the biodegradation of poly(γ-butyrolactone) (PyBL) have been made and remain under development¹³⁻¹⁵. However, up until now most of the research on these strategies has focused on plastics which are less common in daily life.¹⁸

At present, chemically stable and non-biodegradable polyolefins such as PE and polypropylene (PP) represent a large proportion of the polyolefins present in waste plastics. However, a feasible closed loop recycling route for these polyolefins under mild conditions has previously not been established. One major challenge is the selective cleavage of strong C—C bonds in PE and PP at relatively mild conditions¹⁹. High temperature pyrolysis of polyolefins is one way of overcoming the strong C—C bonds but lacks product selectivity and requires significant energy consumption²⁰. Promotion with noble metals such as Pt or Rh have also been used for catalytic pyrolysis but is not considered feasible due to the high costs associated with noble metals. Thus, there remains a need for a viable closed loop recycling strategy for converting waste plastics into valuable products at mild conditions.

The present invention was devised with the foregoing in mind.

### SUMMARY OF THE INVENTION

According to an aspect of the present invention there is provided a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm.

The process for the catalytic decomposition of a polyolefin may be a process for the preparation of a C₃ compound (e.g. propane and/or propene), e.g. by polyolefin decomposition. For example, the process for the catalytic decomposition of a polyolefin may be a process for the preparation of propane, e.g. by polyolefin decomposition. Alternatively, the process for the catalytic decomposition of a polyolefin may be a process for the preparation of propene.

### DETAILED DESCRIPTION OF THE INVENTION

The term “(m-nC)” or “(m-nC) group” used alone or as a prefix, refers to any group having m to n carbon atoms.

The term “alkyl” as used herein refers to straight or branched chain alkyl moieties, typically having 1, 2, 3, 4 or 5 carbon atoms. This term includes reference to groups such as methyl, ethyl, propyl (n-propyl or isopropyl), butyl (n-butyl, sec-butyl or tert-butyl), pentyl and the like. Most suitably, an alkyl may have 1, 2 or 3 carbon atoms.

The term “alkenyl” as used herein refers to straight or branched chain alkenyl moieties, typically having 2, 3, 4 or 5 carbon atoms. The term includes reference to alkenyl moieties typically containing 1 or 2 carbon-carbon double bonds (C═C). This term includes reference to groups such as ethenyl (vinyl), propenyl (allyl), butenyl and pentenyl, as well as both the cis and trans isomers thereof.

The term “alkynyl” as used herein refers to straight or branched chain alkynyl moieties, typically having 2, 3, 4 or 5 carbon atoms. The term includes reference to alkynyl moieties typically containing 1 or 2 carbon-carbon triple bonds (C═C). This term includes reference to groups such as ethynyl, propynyl, butynyl and pentynyl.

The term “aromaticity” as used herein refers to the presence of a an aromatic ring system typically comprising 6, 7, 8, 9 or 10 ring carbon atoms. An aromatic ring system is often phenyl, but may be a polycyclic ring system having two fused rings, at least one of which is aromatic. This term includes reference to groups such as phenyl, naphthyl and the like.

The term “heteroaromaticity” as used herein refers to the presence of an aromatic ring system incorporating one or more (e.g., 1, 2 or 3) ring heteroatoms selected from nitrogen, oxygen and sulfur. A heteroaromatic ring system is often monocyclic, but may be a polycyclic ring system having two fused rings, at least one of which is heteroaromatic. Typically, the heteroaromatic ring system is a 5- or 6-membered ring. Typically, the heteroaromatic ring system will contain up to 3 ring heteroatoms (e.g., nitrogen), more usually up to 2, for example a single ring heteroatom.

The term “substituted” as used herein in reference to a moiety means that one or more (e.g., 1, 2, 3 or 4) of the hydrogen atoms in said moiety are replaced independently of each other by the corresponding number of the described substituents. The term “optionally substituted” as used herein means substituted or unsubstituted.

It will, of course, be understood that substituents are only at positions where they are chemically possible, the person skilled in the art being able to decide (either experimentally or theoretically) without inappropriate effort whether a particular substitution is possible.

Throughout the entirety of the description and claims of this specification, where subject matter is described herein using the term “comprise” (or “comprises” or “comprising”), the same subject matter instead described using the term “consist of” (or “consists of” or “consisting of”) or “consist essentially of” (or “consists essentially of” or “consisting essentially of”) is also contemplated.

Throughout the entirety of the description and claims of this specification, the singular encompasses the plural unless the context otherwise requires. In particular, where the indefinite article is used, the specification is to be understood as contemplating plurality as well as singularity, unless the context requires otherwise.

Features described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any of the specific embodiments recited herein. The invention extends to any novel one, or any novel combination, of the features disclosed in this specification (including any accompanying claims, abstract and drawings), or to any novel one, or any novel combination, of the steps of any method or process so disclosed.

As described hereinbefore, an aspect of the invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm.

Through rigorous investigations, the inventors have devised a vastly improved process for the decomposition of polyolefins found in waste plastics, such as HDPE, LDPE and high density polypropylene (HDPP). In particular, the inventors have found that the presence of an aromatic compound and particular zeolites under an inert atmosphere leads to an efficient, selective and recyclable process for converting waste plastics into more valuable products, such as C1-C4 compounds (i.e. compounds having 1-4 carbon atoms), preferably C3 compounds (i.e. compounds having 3 carbon atoms, such as propane and propene). Furthermore, the inventors have found that not only can the process of the present invention improve the selectivity of polyolefin decomposition to more valuable products, the process also offers advantages in terms of recyclability and costs, indicating that the process of the present invention is an industrially viable route for converting waste plastics into more valuable products.

### Polyolefin

It will be understood that the term “polyolefin” used herein refers to a polymer comprising repeating units formed from the polymerisation of olefin monomers. The polyolefin may therefore comprise repeating units formed from the polymerisation of ethylene monomers, propylene monomers, ethylene terephthalate monomers, vinyl chloride monomers, styrene monomers, or a combination of two or more thereof. Thus, the polyolefin may comprise PE, PP, PET, polyvinyl chloride (PVC), polystyrene (PS), or a combination of two or more thereof. As discussed hereinbefore, the process of the present invention is particularly useful in converting waste plastics into more valuable products, such as gaseous C1-C4 compounds, preferably gaseous C3 compounds. Accordingly, the polyolefin may be provided in the form of a plastic (e.g., a waste plastic). Thus, the present invention also provides a process for the catalytic decomposition of a polyolefin provided in the form of a plastic, wherein the process comprises the step of contacting a polyolefin with a zeolite and an aromatic compound in accordance with the aspect of the present invention. Suitably, the plastic is a waste plastic.

The polyolefin may comprise greater than 60 wt % of PE, PP or a combination thereof. Suitably, the polyolefin comprises greater than 70 wt % of PE, PP or a combination thereof. More suitably, the polyolefin comprises greater than 80 wt % of PE, PP or a combination thereof. Yet more suitably, the polyolefin comprises greater than 90 wt % of PE, PP or a combination thereof. Yet even more suitably, the polyolefin comprises greater than 95 wt % of PE, PP or a combination thereof. Yet still even more suitably, the polyolefin comprises greater than 99 wt % of PE, PP or a combination thereof. In embodiments wherein the polyolefin comprises greater than 60 wt %, 70 wt %, 80 wt %, 90 wt %, 95 wt % or 99 wt % PE, PP or a combination thereof, the remainder of the polyolefin may comprise other repeating units. For example, the remainder of the polyolefin may comprise repeating units formed from the polymerisation of vinyl chloride, styrene, ethylene terephthalate, phenol, formaldehyde, ethylene glycol, acetonitrile or a combination of two or more thereof.

In particular embodiments, the polyolefin is PE, PP or a combination thereof (e.g., the polyolefin consists of PE, PP or a combination thereof). It will be understood that in embodiments wherein the polyolefin comprises a combination of PE and PP, the combination may be in the form of a physical mixture (i.e., a sample comprising both PE and PP) or a copolymer (i.e., block, random or alternate copolymer) of PE and PP.

It will be further understood that the term “polyolefin” used herein encompasses modified polyolefins such as cross-linked polyolefins (e.g., cross-linked polyethylene (PEX)) and ethylene propylene diene monomer (EPDM) rubber) and branched polyolefins.

In certain embodiments, the polyolefin is selected from the group consisting of HDPE, LDPE, linear low-density polyethylene (LLDPE), HDPP, low density polypropylene (LDPP), linear low-density polypropylene (LLDPP) and a combination of two or more thereof. Suitably, the polyolefin is selected from the group consisting of HDPE, LDPE, HDPP and a combination of two or more thereof. In an embodiment, the polyolefin is selected from the group consisting of HDPE, LDPE, HDPP and LDPE/HDPP/HDPP.

### Zeolite

Through extensive investigations, the inventors have found that in the catalytic decomposition of a polyolefin, the presence of particular zeolites significantly improves the effects of the aromatic compound. Without wishing to be bound by theory, the inventors have hypothesised that, during decomposition of a polyolefin, alkylation of the polyolefin with the aromatic compound, followed by subsequent β-scission of a C—C bond to form an alkyl-substituted aromatic compound is promoted when conducted over Brønsted acid sites (BAS) of the zeolite. It is believed that the alkyl scavenging ability of the aromatic compound is significantly augmented when the zeolite has a certain pore size, as well as a plurality of BAS, thereby leading to the efficient and selective decomposition of polyolefins in waste plastic. The zeolite may be considered a catalyst in the process of the invention.

The zeolite may be of the MFI framework type. The phrase “MFI framework” used in the context of zeolites is known in the art and will be understood to mean an aluminosilicate compound (i.e., a compound comprising Al, Si and O atoms) that belongs to structure code MFI defined by the International Zeolite Association (IZA). The typical MFI framework is a corrugated sheet-like structure and comprises a plurality of pores which are defined by the number of ring atoms forming the perimeter of each pore. Suitably, the plurality of pores of the zeolite comprise 10-membered ring channels. Furthermore, it may be that the zeolite is doped (i.e., one more dopant atoms replace an Al, Si and/or O atom in the zeolite framework). Suitably, the zeolite is doped with one or more atoms selected from the group consisting of B and N. More suitably, the zeolite is of the MFI framework type and is doped with one or more atoms selected from the group consisting of B and N.

As specified in an aspect of the present invention, the zeolite comprises a plurality of BAS and a plurality of pores each having a diameter of 0.45-0.60 nm. Due to the diameter of each of the pores, the zeolite may be considered to be microporous (i.e., comprising pores with pore diameters less than 2 nm). Suitably, the plurality of pores of the zeolite each have a diameter of 0.46-0.59 nm. More suitably, the plurality of pores of the zeolite each have a diameter of 0.48-0.58 nm. Yet more suitably, the plurality of pores of the zeolite each have a diameter of 0.50-0.57 nm. Yet even more suitably, the plurality of pores of the zeolite each have a diameter of 0.52-0.56 nm. In a particularly preferred embodiment, the plurality of pores of the zeolite each have a diameter of 0.54-0.56 nm.

Without wishing to be bound by theory, it is believed that the size of the pores of the zeolite plays a key role in promoting the selective formation of gaseous C1-C4 products, particularly gaseous C3 products. In particular, the pore size and geometry of the zeolite appears to be important in determining whether the aromatic compound can access the BAS, which are preferably located in the pores of the zeolite. In a preferred embodiment, the BAS are located in the plurality of pores. The inventors have hypothesised that the BAS of the zeolite are the location of polyolefin decomposition (i.e., the active site), which may proceed via a “hydrocarbon pool” mechanism wherein the aromatic compound, once at the BAS, scavenges alkyl moieties from the polyolefin to form an alkyl-substituted aromatic compound in the pores of the zeolite. The presence of the alkyl-substituted aromatic compounds in the pores of the zeolites resulting in the formation of a “hydrocarbon pool” is thought to be advantageous in a number of ways. First, the alkyl-substituted aromatic compounds can be converted to gaseous C3 products, such as propane (e.g., by cracking), which are more valuable products when compared to the starting waste plastic. Second, the alkyl-substituted aromatic compounds which make up the “hydrocarbon pool” formed in the pores of the zeolite can act as a source of aromatic compound as defined herein. This means that the process of the present invention can continue to proceed, upon addition of more polyolefin, without the need to add more aromatic compound, since the “hydrocarbon pool” formed in the pores of the zeolite can act as a source of aromatic compound. Thus, the specific pore size of the zeolite and presence of the BAS advantageously create a recyclable process for polyolefin decomposition.

The zeolite may comprise a Brunauer-Emmett-Teller (BET) surface area of 200-400 m²/g. Suitably, the zeolite has a BET surface area of 250-350 m²/g. More suitably, the zeolite has a BET surface area of 275-325 m²/g. The zeolite may have a micropore area of 100-300 m²/g. Suitably, the zeolite has a micropore area of 150-250 m²/g. More suitably, the zeolite has a micropore area of 190-230 m²/g. Yet more suitably, the zeolite has a micropore area of 200-220 m²/g.

As will be clear from the above discussion, the acidity of the zeolite is of importance in the process of the present invention. In particular, the presence of a plurality of BAS in/on the zeolite, which can be controlled by the SiO₂/Al₂O₃ ratio (i.e., the molar ratio of Si/Al atoms), promotes the selective decomposition of polyolefins. Suitably, the zeolite has a SiO₂/Al₂O₃ ratio of 10-200. More suitably, the zeolite has a SiO₂/Al₂O₃ ratio of 15-150. Yet more suitably, the zeolite has a SiO₂/Al₂O₃ ratio of 20-125. Yet more suitably, the zeolite has a SiO₂/Al₂O₃ ratio of 25-100. Yet even more suitably, the zeolite has a SiO₂/Al₂O₃ ratio of 30-75.

The zeolite may be a Zeolite Socony Mobil (ZSM)-type zeolite which is acidified (i.e., the zeolite comprises a plurality of BAS). Suitably, the zeolite is selected from the group consisting of ZSM-5, ZSM-11, ZSM-22, ZSM-23 and ZSM-35, each of which having a plurality of BAS. The presence of a plurality of BAS in/on the zeolite may be indicated by the prefix “H” (i.e., H-ZSM-5, H-ZSM-11, H-ZSM-22 etc.) to denote that the zeolite comprises a plurality of BAS. It will be understood that additional charge-balancing ions, such as metal cations (e.g., Na⁺), may also form part of the zeolite framework. Accordingly, the zeolite may be selected from the group consisting of H-ZSM-5, H-ZSM-11, H-ZSM-22, H-ZSM-23 and H-ZSM-35 (where H denotes that the zeolite comprises a plurality of BAS). Suitably, the zeolite is H-ZSM-5. In a preferred embodiment, the zeolite is H-ZSM-5 and comprises a plurality of pores each having a diameter of 0.45-0.60 nm.

The zeolite may be used in an activated (e.g. degassed) form. The zeolite may be activated by thermally treating it (e.g. a temperature of 300-600° C.) under an inert atmosphere (e.g. under nitrogen).

### Aromatic Compound

The presence of an aromatic compound in the process of the present invention leads to an efficient, selective and recyclable process for converting waste plastics into more valuable products. The inventors have hypothesised that the aromatic compound operates by first alkylating the polyolefin to be decomposed followed by subsequent β-scission of a C—C bond to form an alkyl-substituted aromatic compound. It is believed that alkylation of the polyolefin boosts the rate of C—C bond cleavage in the polyolefin at mild conditions, suggesting that the aromatic compound can act as a molecular tweezer by scavenging alkyl moieties from the polyolefin.

The aromatic compound may have a molecular weight of less than 250 g mol⁻¹. Suitably, the aromatic compound has a molecular weight of less than 225 g mol⁻¹. More suitably, the aromatic compound has a molecular weight of less than 200 g mol⁻¹. Yet more suitably, the aromatic compound has a molecular weight of less than 175 g mol⁻¹. Yet even more suitably, the aromatic compound has a molecular weight of less than 150 g mol⁻¹.

An aromatic compound will be understood as being a compound having aromaticity or heteroaromaticity as defined hereinbefore. Aromatic compounds described herein comprise an aromatic or heteroaromatic ring system that is substituted (e.g., with 1, 2, 3 or 4 substituents) or unsubstituted. Suitably, the aromatic compound is a monocyclic aromatic ring (e.g., benzene) or a bicyclic aromatic ring system (e.g., naphthalene), any ring of which is optionally substituted. Possible substituents that may be present are (1-5C) alkyl, (2-5C) alkenyl and/or (2-5C) alkynyl. Suitably, each substituent is independently (1-3C) alkyl (e.g. methyl and/or isopropyl).

Suitably, the aromatic compound is benzene that is optionally substituted with one or more substituents independently selected from (1-5C) alkyl, (2-5C) alkenyl and (2-5C) alkynyl. More suitably, the aromatic compound is benzene that is optionally substituted with one, two, three or four (1-3C) alkyl substituents. Yet more suitably, the aromatic compound benzene that is optionally substituted with one, two, three or four substituents independently selected from methyl, ethyl and isopropyl. In embodiments, the aromatic compound is benzene that is optionally substituted with one, two, three or four substituents independently selected from methyl and isopropyl.

In particular embodiments, the aromatic compound is selected from the group consisting of benzene, toluene, xylene, cumene, mesitylene, 1,2,4,5-tetramethyl benzene and naphthalene. Most suitably, the aromatic compound is selected from the group consisting of benzene, toluene, xylene, cumene, mesitylene and 1,2,4,5-tetramethyl benzene.

### Reaction Conditions

The process of the present invention can be conducted at relatively mild conditions when compared to known processes, which typically consume a vast amount of energy due to the high temperatures traditionally required for polyolefin decomposition. While it is known that the use of noble metal promoters, such as Pt or Ru, can allow such processes to proceed at lower temperatures, the high cost associated with the use of noble metals means that this is not a viable process for largescale polyolefin decomposition from waste plastics. Thus, the process of the invention may be conducted without (i.e. in the absence of) a metal promoter. Suitably, the process of the invention is conducted without (i.e. in the absence of) a noble metal promoter.

Owing to the synergistic benefits of the zeolite and aromatic compound, it is possible for the reaction to proceed at temperatures well below those typical for polyolefin decomposition. The step of contacting the polyolefin with the zeolite and the aromatic compound may be conducted at a temperature of 150-300° C. Suitably, the step of contacting the polyolefin with the zeolite and the aromatic compound is conducted at a temperature of 175-300° C. More suitably, the step of contacting the polyolefin with the zeolite and the aromatic compound is conducted at a temperature of 200-300° C. Yet more suitably, the step of contacting the polyolefin with the zeolite and the aromatic compound is conducted at a temperature of 225-300° C. Yet even more suitably, the step of contacting the polyolefin with the zeolite and the aromatic compound is conducted at a temperature of 250-300° C. In particularly suitable embodiments, the step of contacting the polyolefin with the zeolite and the aromatic compound is conducted at a temperature of 250-280° C.

Suitably, the weight ratio of the zeolite to the aromatic compound is 1:(0.1-10). More suitably, the weight ratio of the zeolite to the aromatic compound is 1:(0.5-5).

The step of contacting a polyolefin with a zeolite and an aromatic compound is conducted under an inert atmosphere comprising hydrogen. It is hypothesised that the presence of hydrogen in an inert atmosphere mitigates the risk of zeolite deactivation. Thus, it may be that the step of contacting the polyolefin with the zeolite and the aromatic compound is conducted in an atmosphere of hydrogen. Suitably, the step of contacting the polyolefin with the zeolite and the aromatic compound is conducted in an atmosphere of 10-50 bar hydrogen. More suitably, the step of contacting the polyolefin with the zeolite and the aromatic compound is conducted in an atmosphere of 20-40 bar hydrogen. Yet more suitably, the step of contacting the polyolefin with the zeolite and the aromatic compound is conducted in an atmosphere of 25-35 bar hydrogen. In embodiments, the step of contacting the polyolefin with the zeolite and the aromatic compound is conducted in an atmosphere of 30 bar hydrogen. The inert atmosphere may comprise another inert gas, such as argon and/or nitrogen. In certain embodiments, the step of contacting the polyolefin with the zeolite and the aromatic compound may be conducted in an atmosphere of nitrogen and hydrogen.

In view of the above and in accordance with an aspect of the present invention, the process of catalytically decomposing a polyolefin may additionally comprise the steps of:
(1) dehydrogenating the polyolefin to form an alkene (i.e., a new C═C double bond in the polyolefin); and
(2) protonating said alkene to form a carbocation.

Suitably, steps (1) and (2) take place at the BAS of the zeolite. More suitably, steps (1) and (2) take place at the BAS, which are located in the pores of the zeolite.

It is hypothesised that once step (2) is complete (i.e., a carbocation has been formed), the aromatic compound binds to the carbocation site. The presence of the aromatic compound on the modified polyolefin is believed to induce β-scission of a C—C bond in the polyolefin to form an alkyl-substituted aromatic compound and a fragmented (i.e., reduced chain length) polyolefin. The alkyl-substituted aromatic compound can be converted into useful products such as gaseous C3 compounds and/or used as part of the “hydrocarbon pool” mechanism as a source of aromatic compound as defined herein. Thus, the process of catalytically decomposing a polyolefin may additionally comprise the steps of:
(3) alkylating said carbocation with an aromatic compound to form a polyolefin-aromatic compound complex; and
(4) cleaving a C—C bond of the polyolefin-aromatic compound complex.

Suitably, steps (3) and (4) take place at the BAS of the zeolite. More suitably, steps (3) and (4) take place at the BAS, which are located in the pores of the zeolite.

### Particular Embodiments

The following paragraphs describe certain embodiments of the process of the present invention. Unless clearly incompatible therewith, it will be understood that the following embodiments may be taken in combination with any other feature of the invention hitherto described.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 80 wt % of PE, PP or a combination thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 95 wt % of PE, PP or a combination thereof;
the zeolite is of the MFI framework type; and
the aromatic compound has a molecular weight of less than 250 g mol⁻¹.

In a preferred embodiment, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin is selected from the group consisting of HDPE, LDPE, LLDPE, HDPP, LDPP, LLDPP and a combination of two or more thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.48-0.58 nm; and
   wherein:
the polyolefin comprises greater than 60 wt % of PE, PP or a combination thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.52-0.56 nm; and
   wherein:
the polyolefin comprises greater than 60 wt % of PE, PP or a combination thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof;
the zeolite is selected from the group consisting of ZSM-5, ZSM-11, ZSM-22, ZSM-23 and ZSM-35, each of which having a plurality of BAS; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof;
the zeolite is of the MFI framework type; and
the aromatic compound is benzene that is optionally substituted with one or more substituents independently selected from (1-5C) alkyl, (2-5C) alkenyl and (2-5C) alkynyl.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof;
the zeolite is of the MFI framework type; and
the aromatic compound is benzene that is optionally substituted with one, two, three or four (1-3C) alkyl substituents.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof;
the zeolite is of the MFI framework type; and
the aromatic compound is selected from the group consisting of benzene, toluene, xylene, cumene, mesitylene, 1,2,4,5-tetramethyl benzene and naphthalene.

In certain embodiments, the present invention provides a process for the catalytic decomposition of a polyolefin, the process comprising a step of contacting a polyolefin with:
(a) a zeolite, and
(b) an aromatic compound
   at a temperature of 200-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 250-300° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 250-280° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:

(a) a zeolite, and

(b) an aromatic compound

at a temperature of 150-300° C. and under an inert atmosphere comprising 10-50 bar hydrogen;
wherein the zeolite comprises a plurality of Brønsted acid sites and a plurality of pores each having a diameter of 0.45-0.60 nm; and
wherein:
the polyolefin comprises greater than 60 wt % of PE, PP or a combination thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 150-300° C. and under an inert atmosphere comprising 20-40 bar hydrogen;
   wherein the zeolite comprises a plurality of Brønsted acid sites and a plurality of pores each having a diameter of 0.45-0.60 nm; and
   wherein:
the polyolefin comprises greater than 60 wt % of PE, PP or a combination thereof;
the zeolite is of the MFI framework type; and
the aromatic compound has a molecular weight of less than 250 g mol⁻¹.

In certain embodiments, the present invention provides a process for the catalytic decomposition of a polyolefin, the process comprising a step of contacting a polyolefin with:
(a) a zeolite, and
(b) an aromatic compound
   at a temperature of 200-300° C. and under an inert atmosphere comprising hydrogen;
   wherein the zeolite comprises a plurality of Brønsted acid sites and a plurality of pores each having a diameter of 0.48-0.58 nm; and
   wherein:
the polyolefin comprises greater than 80 wt % of PE, PP or a combination thereof;
the zeolite is of the MFI framework type; and
the aromatic compound is benzene that is optionally substituted with one or more substituents independently selected from (1-5C) alkyl, (2-5C) alkenyl and (2-5C) alkynyl.

In certain embodiments, the present invention provides a process for the catalytic decomposition of a polyolefin, the process comprising a step of contacting a polyolefin with:
(a) a zeolite, and
(b) an aromatic compound
   at a temperature of 250-300° C. and under an inert atmosphere comprising hydrogen;
   wherein the zeolite comprises a plurality of Brønsted acid sites and a plurality of pores each having a diameter of 0.52-0.58 nm; and
   wherein:
the polyolefin comprises greater than 95 wt % of PE, PP or a combination thereof;
the zeolite is of the MFI framework type; and
the aromatic compound is benzene and is optionally substituted with one, two, three or four (1-3C) alkyl substituents.

In certain embodiments, the present invention provides a process for the catalytic decomposition of a polyolefin, the process comprising a step of contacting a polyolefin with:
(a) a zeolite, and
(b) an aromatic compound
   at a temperature of 250-280° C. and under an inert atmosphere comprising hydrogen;
   wherein the zeolite comprises a plurality of Brønsted acid sites and a plurality of pores each having a diameter of 0.45-0.60 nm; and
   wherein:
the polyolefin is selected from the group consisting of HDPE, LDPE, LLDPE, HDPP, LDPP, LLDPP and a combination of two or more thereof;
the zeolite is selected from the group consisting of ZSM-5, ZSM-11, ZSM-22, ZSM-23 and ZSM-35, each of which having a plurality of BAS; and
the aromatic compound is selected from the group consisting of benzene, toluene, xylene, cumene, mesitylene, 1,2,4,5-tetramethyl benzene and naphthalene.

The following numbered statements 1 to 93 are not claims, but instead describe particular aspects and embodiments of the invention:
1. A process for the catalytic decomposition of a polyolefin, the process comprising a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm.
2. The process of statement 1, wherein the polyolefin comprises repeating units formed from the polymerisation of ethylene monomers, propylene monomers, ethylene terephthalate monomers, vinyl chloride monomers, styrene monomers, or a combination of two or more thereof.
3. The process of statement 1 or 2, wherein the polyolefin comprises PE, PP, PET, polyvinyl chloride (PVC), polystyrene (PS), or a combination of two or more thereof. 4. The process of statement 1, 2 or 3, wherein the polyolefin is provided in the form of a plastic.
5. The process of any one of the preceding statements, wherein the polyolefin is provided in the form of a waste plastic.
6. The process of any one of the preceding statements, wherein polyolefin comprises greater than 60 wt % of PE, PP or a combination thereof.
7. The process of any one of the preceding statements, wherein the polyolefin comprises greater than 70 wt % of PE, PP or a combination thereof.
8. The process of any one of the preceding statements, wherein the polyolefin comprises greater than 80 wt % of PE, PP or a combination thereof.
9. The process of any one of the preceding statements, wherein the polyolefin comprises greater than 90 wt % of PE, PP or a combination thereof.
10. The process of any one of the preceding statements, wherein the polyolefin comprises greater than 95 wt % of PE, PP or a combination thereof.
11. The process of any one of the preceding statements, wherein the polyolefin comprises greater than 99 wt % of PE, PP or a combination thereof.
12. The process of any one of the preceding statements, wherein the polyolefin is PE, PP or a combination thereof.
13. The process of any one of the preceding statements, wherein the polyolefin is selected from the group consisting of HDPE, LDPE, LLDPE, HDPP, LDPP, LLDPP and a combination of two or more thereof.
14. The process of any one of the preceding statements, wherein the polyolefin is selected from the group consisting of HDPE, LDPE, HDPP and a combination of two or more thereof.
15. The process of any one of the preceding statements, wherein the polyolefin is selected from the group consisting of HDPE, LDPE, HDPP and LDPE/HDPP/HDPP.
16. The process of any one of the preceding statements, wherein the zeolite is of the MFI framework type.
17. The process of any one of the preceding statements, wherein the plurality of pores of the zeolite comprise 10-membered ring channels.
18. The process of any one of the preceding statements, wherein the plurality of pores of the zeolite each have a diameter of 0.46-0.59 nm.
19. The process of any one of the preceding statements, wherein the plurality of pores of the zeolite each have a diameter of 0.48-0.58 nm.
20. The process of any one of the preceding statements, wherein the plurality of pores of the zeolite each have a diameter of 0.50-0.57 nm.
21. The process of any one of the preceding statements, wherein the plurality of pores of the zeolite each have a diameter of 0.52-0.56 nm.
22. The process of any one of the preceding statements, wherein the plurality of pores of the zeolite each have a diameter of 0.54-0.56 nm.
23. The process of any one of the preceding statements, wherein the BAS are located in the plurality of pores.
24. The process of any one of the preceding statements, wherein the zeolite has a BET surface area of 200-400 m²/g.
25. The process of any one of the preceding statements, wherein the zeolite has a BET surface area of 250-350 m²/g.
26. The process of any one of the preceding statements, wherein the zeolite has a BET surface area of 275-325 m²/g.
27. The process of any one of the preceding statements, wherein the zeolite has a micropore area of 100-300 m²/g.
28. The process of any one of the preceding statements, wherein the zeolite has a micropore area of 150-250 m²/g.
29. The process of any one of the preceding statements, wherein the zeolite has a micropore area of 190-230 m²/g.
30. The process of any one of the preceding statements, wherein the zeolite has a micropore area of 200-220 m²/g.
31. The process of any one of the preceding statements, wherein the zeolite has a SiO₂/Al₂O₃ ratio of 10-200.
32. The process of any one of the preceding statements, wherein the zeolite has a SiO₂/Al₂O₃ ratio of 15-150.
33. The process of any one of the preceding statements, wherein the zeolite has a SiO₂/Al₂O₃ ratio of 20-125.
34. The process of any one of the preceding statements, wherein the zeolite has a SiO₂/Al₂O₃ ratio of 25-100.
35. The process of any one of the preceding statements, wherein the zeolite has a SiO₂/Al₂O₃ ratio of 30-75.
36. The process of any one of the preceding statements, wherein the zeolite is selected from the group consisting of ZSM-5, ZSM-11, ZSM-22, ZSM-23 and ZSM-35, each of which having a plurality of BAS (e.g., the zeolite is selected from the group consisting of H-ZSM-5, H-ZSM-11, H-ZSM-22, H-ZSM-23 and H-ZSM-35).
37. The process of any one of the preceding statements, wherein the zeolite is ZSM-5 having a plurality of BAS.
38. The process of any one of the preceding statements, wherein the zeolite is selected from the group consisting of ZSM-5, ZSM-11, ZSM-22, ZSM-23 and ZSM-35, each of which having a plurality of BAS and the polyolefin is selected from the group consisting of HDPE, LDPE, LLDPE, HDPP, LDPP, LLDPP and a combination of two or more thereof.
39. The process of any one of the preceding statements, wherein the zeolite is ZSM-5 having a plurality of BAS and the polyolefin is selected from the group consisting of HDPE, LDPE, HDPP and a combination of two or more thereof.
40. The process of any one of the preceding statements, wherein the aromatic compound has a molecular weight of less than 250 g mol⁻¹.
41. The process of any one of the preceding statements, wherein the aromatic compound has a molecular weight of less than 225 g mol⁻¹.
42. The process of any one of the preceding statements, wherein the aromatic compound has a molecular weight of less than 200 g mol⁻¹.
43. The process of any one of the preceding statements, wherein the aromatic compound has a molecular weight of less than 175 g mol⁻¹.
44. The process of any one of the preceding statements, wherein the aromatic compound has a molecular weight of less than 150 g mol⁻¹.
45. The process of any one of the preceding statements, wherein the aromatic compound is a monocyclic aromatic ring (e.g., benzene) or a bicyclic aromatic ring system (e.g., naphthalene), any ring of which is optionally substituted with one or more substituents independently selected from (1-5C) alkyl, (2-5C) alkenyl and (2-5C) alkynyl.
46. The process of statement 45, wherein the one or more substituents are 1, 2, 3 or 4 substituents.
47. The process of statement 45 or 46, wherein the one or more substituents are independently selected from (1-4C) alkyl, (2-4C) alkenyl and (2-4C) alkynyl.
48. The process of statement 45 or 46, wherein the one or more substituents are independently selected from (1-4C) alkyl.
49. The process of statement 45 or 46, wherein the one or more substituents are independently selected from (1-3C) alkyl.
50. The process of statement 45 or 46, wherein the one or more substituents are independently selected from methyl, ethyl and isopropyl.
51. The process of statement 45 or 46, wherein the one or more substituents methyl substituents.
52. The process of any one of the preceding statements, wherein the aromatic compound is benzene that is optionally substituted with one or more substituents independently selected from (1-5C) alkyl, (2-5C) alkenyl and (2-5C) alkynyl.
53. The process of any one of the preceding statements, wherein the aromatic compound is benzene that is optionally substituted with one, two, three or four (1-3C) alkyl substituents.
54. The process of any one of the preceding statements, wherein the aromatic compound benzene that is optionally substituted with one, two, three or four substituents independently selected from methyl, ethyl and isopropyl.
55. The process of any one of the preceding statements, wherein the aromatic compound is benzene that is optionally substituted with one, two, three or four substituents independently selected from methyl and isopropyl.
56. The process of any one of the preceding statements, wherein the aromatic compound is selected from the group consisting of benzene, toluene, xylene, cumene, mesitylene, 1,2,4,5-tetramethyl benzene and naphthalene.
57. The process of any one of the preceding statements, wherein the aromatic compound is selected from the group consisting of benzene, toluene, xylene, cumene, mesitylene and 1,2,4,5-tetramethyl benzene.
58. The process of any one of the preceding statements, wherein the step of contacting the polyolefin with the zeolite and the aromatic compound is conducted at a temperature of 175-300° C.
59. The process of any one of the preceding statements, wherein the step of contacting the polyolefin with the zeolite and the aromatic compound is conducted at a temperature of 200-300° C.
60. The process of any one of the preceding statements, wherein the step of contacting the polyolefin with the zeolite and the aromatic compound is conducted at a temperature of 225-300° C.
61. The process of any one of the preceding statements, wherein the step of contacting the polyolefin with the zeolite and the aromatic compound is conducted at a temperature of 250-300° C.
62. The process of any one of the preceding statements, wherein the step of contacting the polyolefin with the zeolite and the aromatic compound is conducted at a temperature of 250-280° C.
63. The process of any one of the preceding statements, wherein the weight ratio of the zeolite to the aromatic compound is 1:(0.1-10).
64. The process of any one of the preceding statements, wherein the weight ratio of the zeolite to the aromatic compound is 1:(0.5-5).
65. The process of any one of the preceding statements, wherein the step of contacting the polyolefin with the zeolite and the aromatic compound is conducted in an atmosphere of hydrogen.
66. The process of any one of the preceding statements, wherein the step of contacting the polyolefin with the zeolite and the aromatic compound is conducted in an atmosphere of 10-50 bar hydrogen.
67. The process of any one of the preceding statements, wherein the step of contacting the polyolefin with the zeolite and the aromatic compound is conducted in an atmosphere of 20-40 bar hydrogen.
68. The process of any one of the preceding statements, wherein the step of contacting the polyolefin with the zeolite and the aromatic compound is conducted in an atmosphere of 25-35 bar hydrogen.
69. The process of any one of the preceding statements, wherein the step of contacting the polyolefin with the zeolite and the aromatic compound is conducted in an atmosphere of 30 bar hydrogen.
70. The process of any one of the preceding statements, wherein the step of contacting the polyolefin with the zeolite and the aromatic compound is conducted in an atmosphere of nitrogen, argon and hydrogen.
71. The process of any one of the preceding statements, wherein the step of contacting the polyolefin with the zeolite and the aromatic compound is conducted in an atmosphere of argon and hydrogen.
72. The process of any one of the preceding statements, wherein the step of contacting the polyolefin with the zeolite and the aromatic compound is conducted in an atmosphere of nitrogen and hydrogen.
73. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof; and
   the aromatic compound has a molecular weight of less than 250 g mol⁻¹.
74. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is of the MFI framework type; and
   the aromatic compound has a molecular weight of less than 250 g mol⁻¹.
75. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 80 wt % of PE, PP or a combination thereof;
   the zeolite is of the MFI framework type; and
   the aromatic compound has a molecular weight of less than 250 g mol⁻¹.
76. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 95 wt % of PE, PP or a combination thereof;
   the zeolite is of the MFI framework type; and
   the aromatic compound has a molecular weight of less than 250 g mol⁻¹.
77. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin is selected from the group consisting of HDPE, LDPE, LLDPE, HDPP, LDPP, LLDPP and a combination of two or more thereof;
   the zeolite is of the MFI framework type; and
   the aromatic compound has a molecular weight of less than 250 g mol⁻¹.
78. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.48-0.58 nm; and
wherein:
   the polyolefin comprises greater than 60 wt % of PE, PP or a combination thereof;
   the zeolite is of the MFI framework type; and
   the aromatic compound has a molecular weight of less than 250 g mol⁻¹.
79. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.52-0.56 nm; and
wherein:
   the polyolefin comprises greater than 60 wt % of PE, PP or a combination thereof;
   the zeolite is of the MFI framework type; and
   the aromatic compound has a molecular weight of less than 250 g mol⁻¹.
80. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is selected from the group consisting of ZSM-5, ZSM-11, ZSM-22, ZSM-23 and ZSM-35, each of which having a plurality of BAS; and
   the aromatic compound has a molecular weight of less than 250 g mol⁻¹.
81. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is of the MFI framework type; and
   the aromatic compound is benzene that is optionally substituted with one or more substituents independently selected from (1-5C) alkyl, (2-5C) alkenyl and (2-5C) alkynyl.
82. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is of the MFI framework type; and
   the aromatic compound is benzene and is optionally substituted with one, two, three or four (1-3C) alkyl substituents.
83. The process of any one of the preceding statements, wherein the process comprises a step of contacting a polyolefin with:
   (a) a zeolite, and
   (b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is of the MFI framework type; and
   the aromatic compound is selected from the group consisting of benzene, toluene, xylene, cumene, mesitylene, 1,2,4,5-tetramethyl benzene and naphthalene.
84. The process of any one of the preceding statements, wherein the process comprises a step of contacting a polyolefin with:
   (a) a zeolite, and
   (b) an aromatic compound
at a temperature of 200-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is of the MFI framework type; and
   the aromatic compound has a molecular weight of less than 250 g mol⁻¹.
85. The process of any one of the preceding statements, wherein the process comprises a step of contacting a polyolefin with:
   (a) a zeolite, and
   (b) an aromatic compound
at a temperature of 250-300° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is of the MFI framework type; and
   the aromatic compound has a molecular weight of less than 250 g mol⁻¹.
86. The process of any one of the preceding statements, wherein the process comprises a step of contacting a polyolefin with:
   (a) a zeolite, and
   (b) an aromatic compound
at a temperature of 250-280° C. and under an inert atmosphere comprising hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is of the MFI framework type; and
   the aromatic compound has a molecular weight of less than 250 g mol⁻¹.
87. The process of any one of the preceding statements, wherein the process comprises a step of contacting a polyolefin with:
   (a) a zeolite, and
   (b) an aromatic compound
at a temperature of 150-300° C. and under an inert atmosphere comprising 10-50 bar hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is of the MFI framework type; and
   the aromatic compound has a molecular weight of less than 250 g mol⁻¹.
88. The process of any one of the preceding statements, wherein the process comprises a step of contacting a polyolefin with:
   (a) a zeolite, and
   (b) an aromatic compound
at a temperature of 150-300° C. and under an inert atmosphere comprising 20-40 bar hydrogen;
wherein the zeolite comprises a plurality of Brønsted acid sites and a plurality of pores each having a diameter of 0.45-0.60 nm; and
wherein:
   the polyolefin comprises greater than 60 wt % of PE, PP or a combination thereof;
   the zeolite is of the MFI framework type; and
   the aromatic compound has a molecular weight of less than 250 g mol⁻¹.
8. The process of any one of the preceding statements, wherein the process comprises a step of contacting a polyolefin with:
   (a) a zeolite, and
   (b) an aromatic compound
at a temperature of 200-300° C. and under an inert atmosphere comprising hydrogen;
wherein the zeolite comprises a plurality of Brønsted acid sites and a plurality of pores each having a diameter of 0.48-0.58 nm; and
wherein:
   the polyolefin comprises greater than 80 wt % of PE, PP or a combination thereof;
   the zeolite is of the MFI framework type; and
   the aromatic compound is benzene that is optionally substituted with one or more substituents independently selected from (1-5C) alkyl, (2-5C) alkenyl and (2-5C) alkynyl.
90. The process of any one of the preceding statements, wherein the process comprises a step of contacting a polyolefin with:
   (a) a zeolite, and
   (b) an aromatic compound
at a temperature of 250-300° C. and under an inert atmosphere comprising hydrogen;
wherein the zeolite comprises a plurality of Brønsted acid sites and a plurality of pores each having a diameter of 0.52-0.58 nm; and
wherein:
   the polyolefin comprises greater than 95 wt % of PE, PP or a combination thereof;
   the zeolite is of the MFI framework type; and
   the aromatic compound is benzene and is optionally substituted with one, two, three or four (1-3C) alkyl substituents.
91. The process of any one of the preceding statements, wherein the process comprises a step of contacting a polyolefin with:
   (a) a zeolite, and
   (b) an aromatic compound
at a temperature of 250-280° C. and under an inert atmosphere comprising hydrogen;
wherein the zeolite comprises a plurality of Brønsted acid sites and a plurality of pores each having a diameter of 0.45-0.60 nm; and
wherein:
   the polyolefin is selected from the group consisting of HDPE, LDPE, LLDPE, HDPP, LDPP, LLDPP and a combination of two or more thereof;
   the zeolite is selected from the group consisting of ZSM-5, ZSM-11, ZSM-22, ZSM-23 and ZSM-35, each of which having a plurality of BAS; and
   the aromatic compound is selected from the group consisting of benzene, toluene, xylene, cumene, mesitylene, 1,2,4,5-tetramethyl benzene and naphthalene.
92. The process of any one of the preceding statements, wherein the zeolite is doped with one or more atoms selected from the group consisting of B and N.
93. The process of any one of the preceding statements, wherein, the zeolite is of the MFI framework type and is doped with one or more atoms selected from the group consisting of B and N.

### EXAMPLES

One or more examples of the invention will now be described, for the purpose of illustration only, with reference to the accompanying figures:

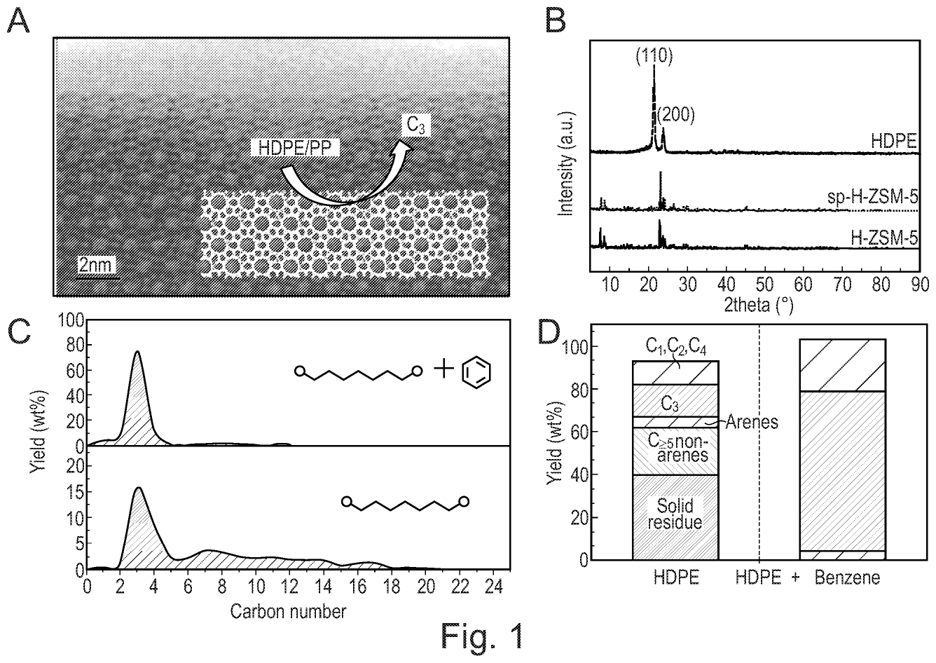

FIG. 1. Benzene promoted HDPE decomposition over HZSM-5.

FIG. 1A. Bright field TEM image of H-ZSM-5 catalyst and a motif exhibiting the framework of HZSM-5 and the reaction of HDPE to C3.

FIG. 1B. XRD patterns of H-ZSM-5 catalyst, HDPE, and spent H-ZSM-5 catalyst.

FIG. 1C. A comparison of product distribution of HDPE decomposition with cofed benzene and without cofed benzene.

FIG. 1D. The mass content of products and solid residue with/without cofed benzene. Reaction condition: 2 g of HDPE, 0.2 g of H-ZSM-5, 280° C., 30 bar H₂ for 10 h with/without 0.2 g of benzene.

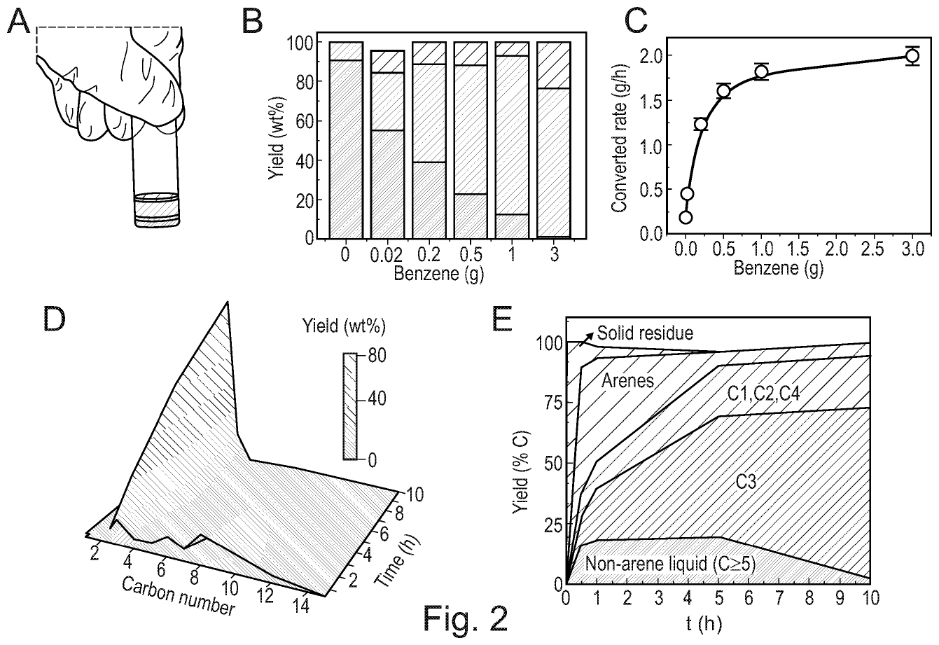

FIG. 2. The effect of benzene on the decomposition of HDPE over H-ZSM-5.

FIG. 2A. The collected products and spent catalyst after centrifugation at 250° C., 30 bar H₂ for 1 h with 0.2 g of benzene and 2 g of HDPE.

FIG. 2B. The mass content of liquid and solid residue as a function of addition of benzene at same condition as FIG. 2A. The benzene weight is excluded from the liquid mass. The black, orange and yellow colours represent solid residue, liquid and gas, respectively.

FIG. 2C. The conversion of HDPE as a function of amount of cofed benzene at same condition as FIG. 2A.

FIG. 2D. A trajectory of carbon product distribution in mass (wt %) during HDPE decomposition over H-ZSM-5 at 280° C., 30 bar H₂ with 0.2 g of benzene and 2 g of HDPE, according to reaction time.

FIG. 2E. The product distribution of benzene promoted degradation of HDPE over H-ZSM-5 based on the molar amount of carbon (% C) for FIG. 2D. Initial benzene is excluded.

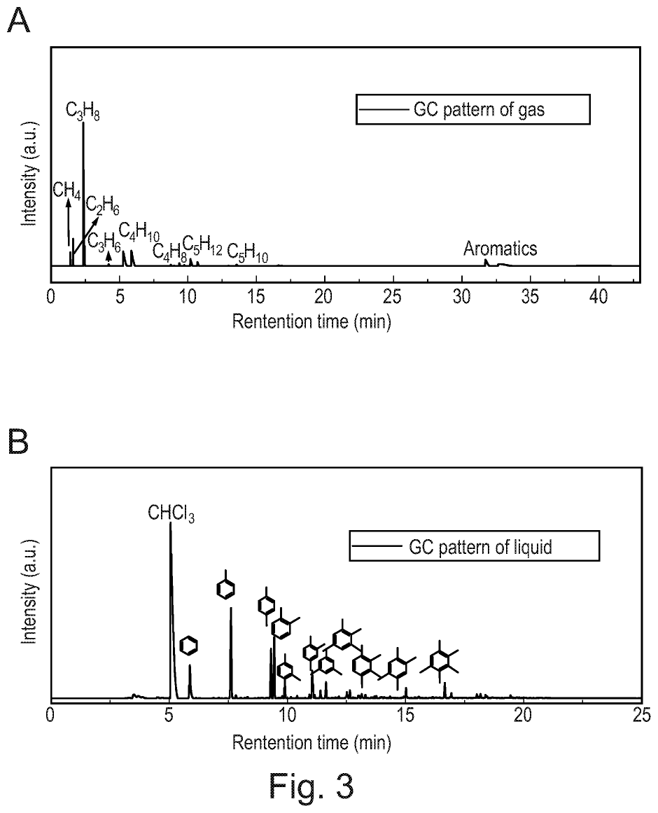

FIG. 3. GC patterns of obtained gas (A) and liquid (B) products. Reaction conditions: 280° C., 10 h, Benzene, 0.2 g.

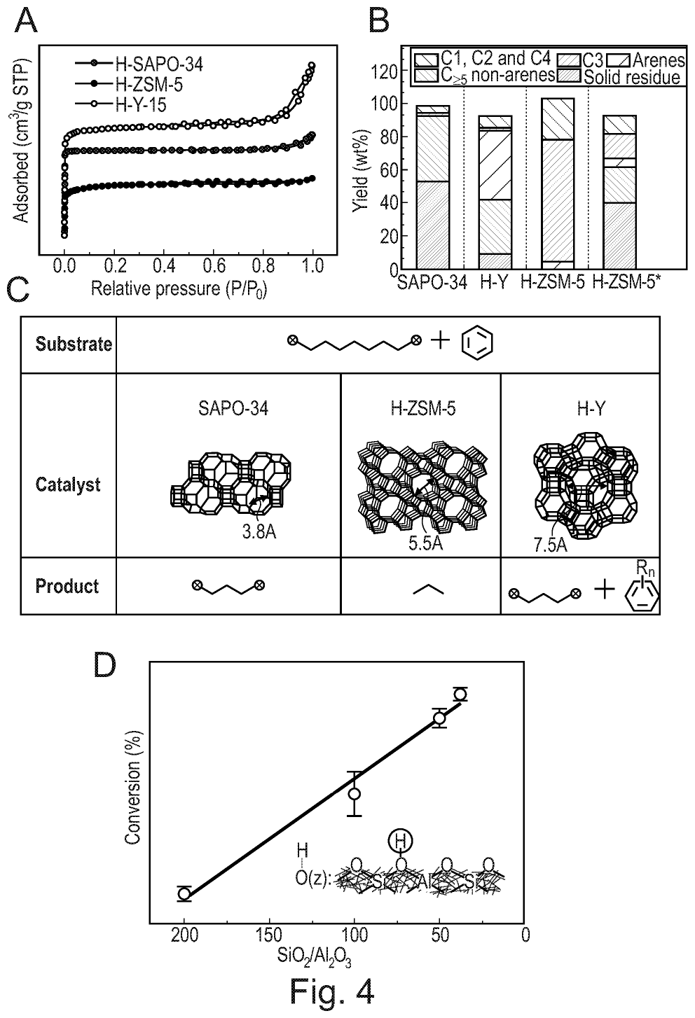

FIG. 4A. N₂ sorption of different zeolite catalysts.

FIG. 4B. Product distribution and yield over different zeolites at 280° C. at 30 bar H₂ for 10 h with 0.2 g of benzene. H-ZSM5* indicates that the reaction proceeded without cofed benzene.

FIG. 4C. Production species over SAPO-34, H-ZSM-5 and H—Y zeolites.

FIG. 4D. HDPE decomposition at 250° C. at 30 bar H₂ for 1 h with 0.2 g of benzene with different SiO₂/Al₂O₃ ratio.

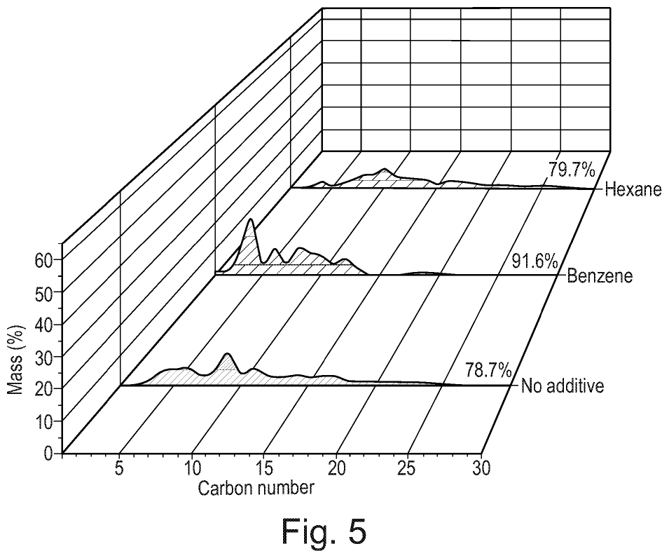

FIG. 5. HDPE (2 g) conversion and mass product distribution over SiO₂—Al₂O₃ catalyst without promoter, with cofed benzene (2 g) and with cofed hexane (2 g) under 30 bar N₂ at 300° C. for 4 h.

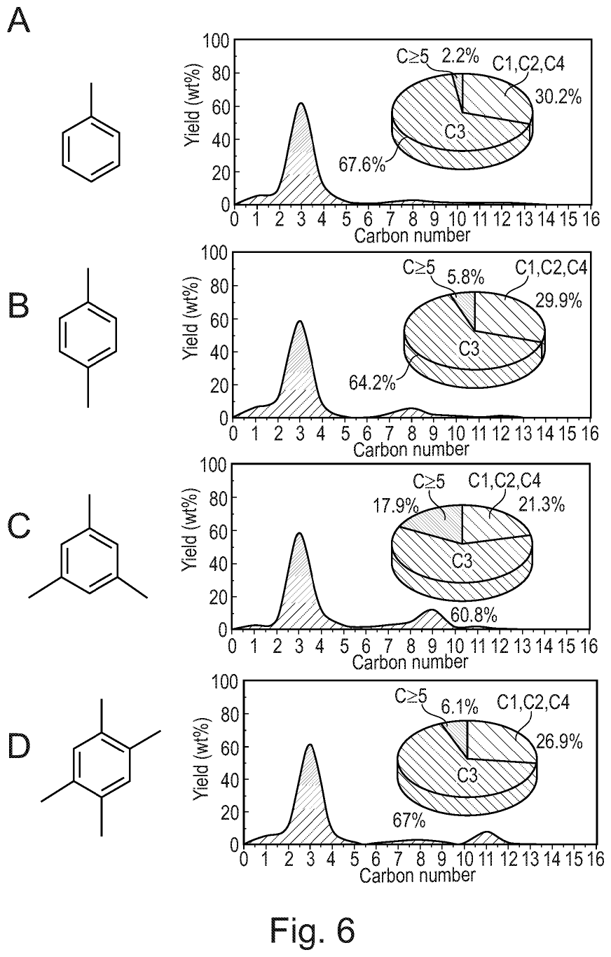

FIG. 6. Product distribution over the H-ZSM-5 catalyst with 0.2 g of cofed (A) toluene, (B) xylene, (C) mesitylene, and (D) 1,2,4,5 methyl benzene giving a complete conversion of 2 g of HDPE under 30 bar of H₂ at 280° C. for 10 h.

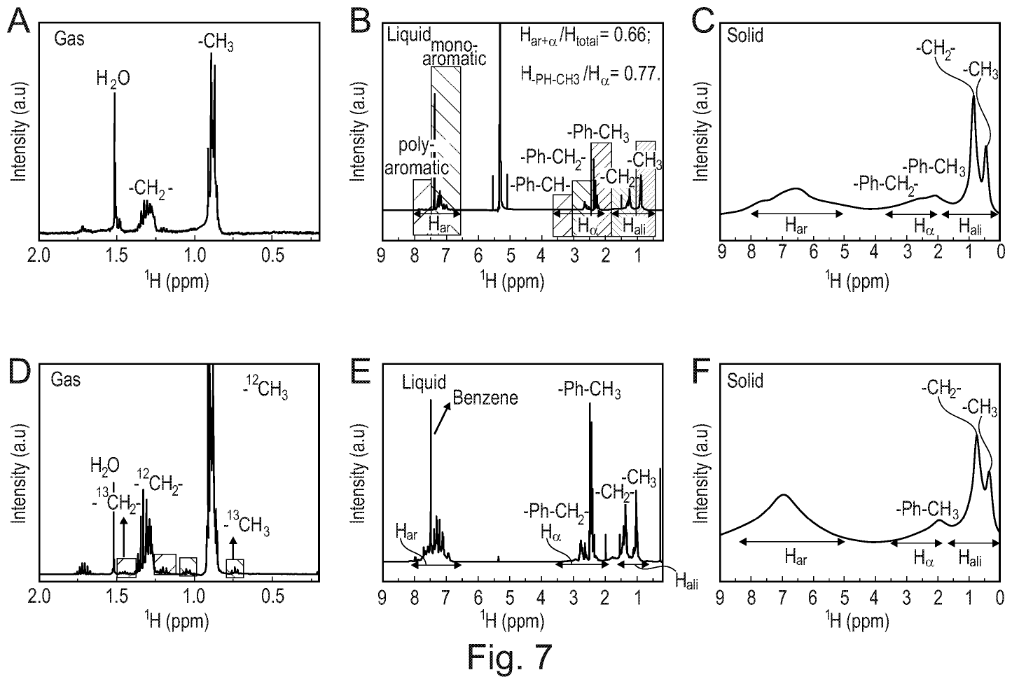

FIG. 7. ¹H NMR of gas, liquid, and solid products from HDPE decomposition with unlabelled benzene (A, B and C) and ¹³C benzene (D, E and F). Reaction condition: HDPE, 2 g, 280° C., 10 h, Benzene, 0.2 g. H₂, 30 bar.

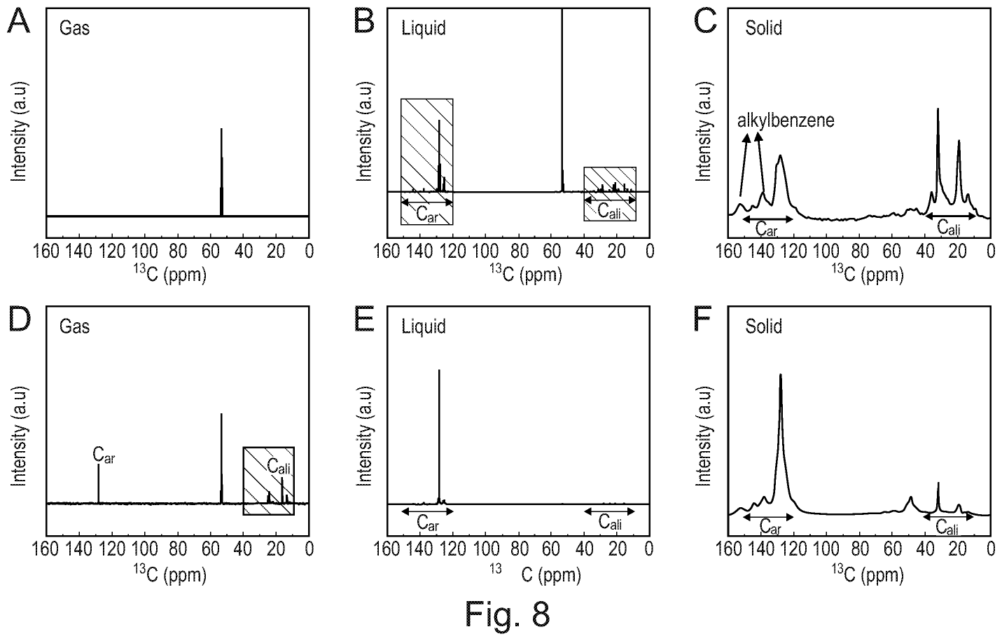

FIG. 8. ¹³C NMR of gas (A and D), liquid (B and E), and solid products (C and F) from HDPE decomposition with unlabelled benzene (A, B and C) and ¹³C benzene (D, E and F). Reaction condition: HDPE, 2 g, 280° C., 10 h, Benzene, 0.2 g. H₂, 30 bar.

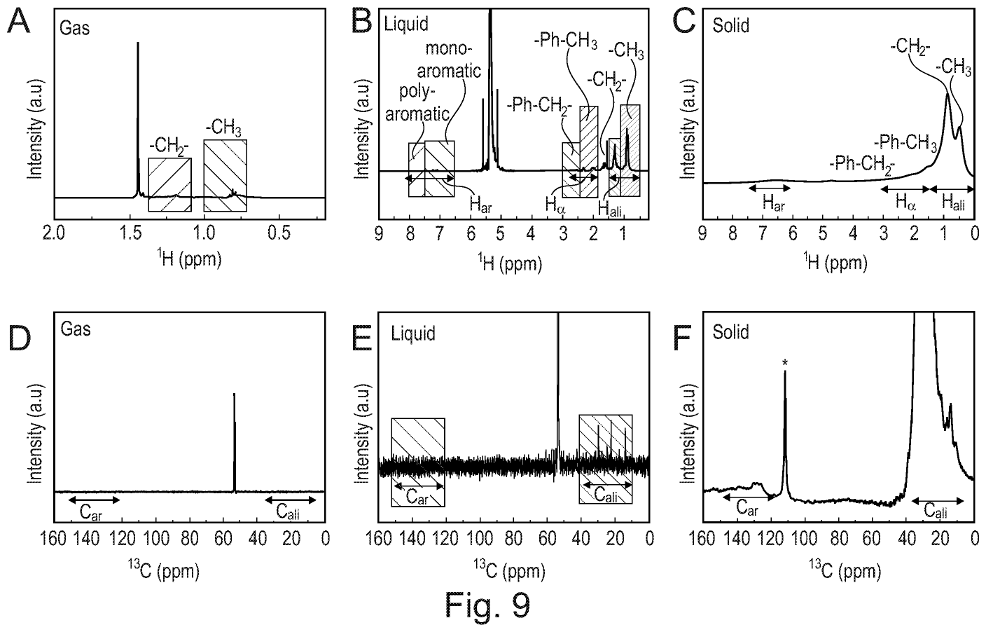

FIG. 9. ¹H NMR (A, B and C) and ¹³C NMR (D, E and F) of gas, liquid, and solid products from HDPE decomposition without benzene. Reaction condition: HDPE, 2 g, 280° C., 10 h, H₂, 30 bar.

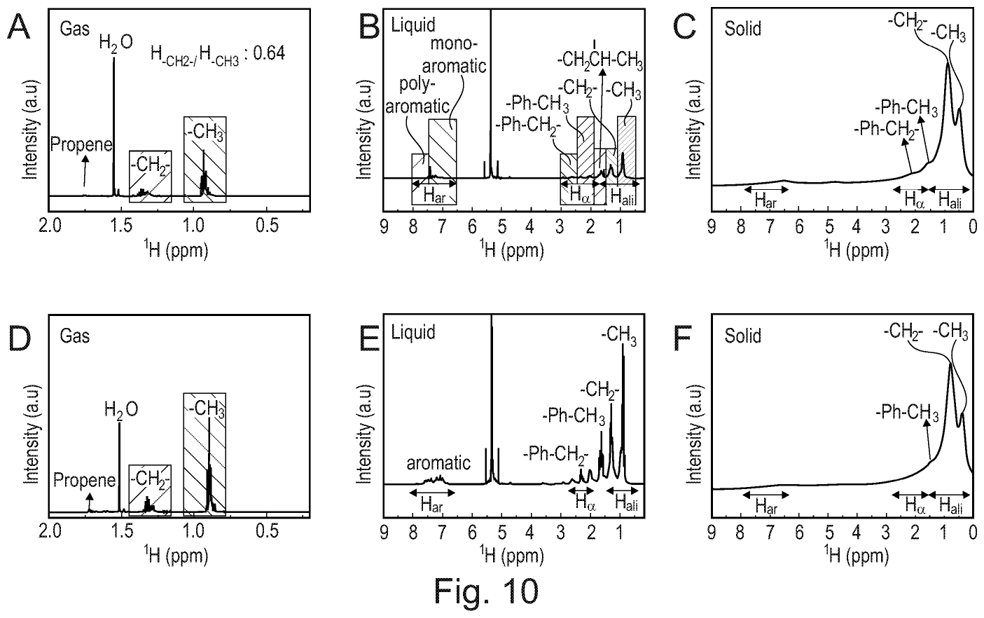

FIG. 10. ¹H NMR of gas, liquid, and solid products from HDPE decomposition with unlabelled benzene (A, B and C) and ¹³C benzene (D, E and F). Reaction condition: HDPE, 2 g, 250° C., 1 h, Benzene, 0.2 g. H₂, 30 bar.

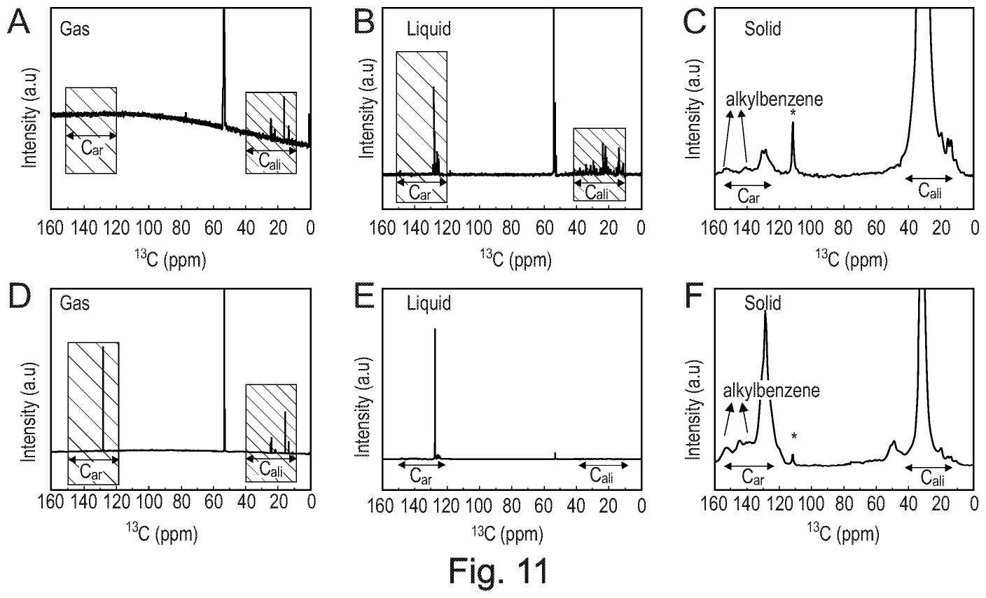

FIG. 11. ¹³C NMR of gas, liquid, and solid products from HDPE decomposition with unlabelled benzene (A, B and C) and ¹³C benzene (D, E and F). Reaction condition: HDPE, 2 g, 250° C., 1 h, Benzene, 0.2 g. H₂, 30 bar.

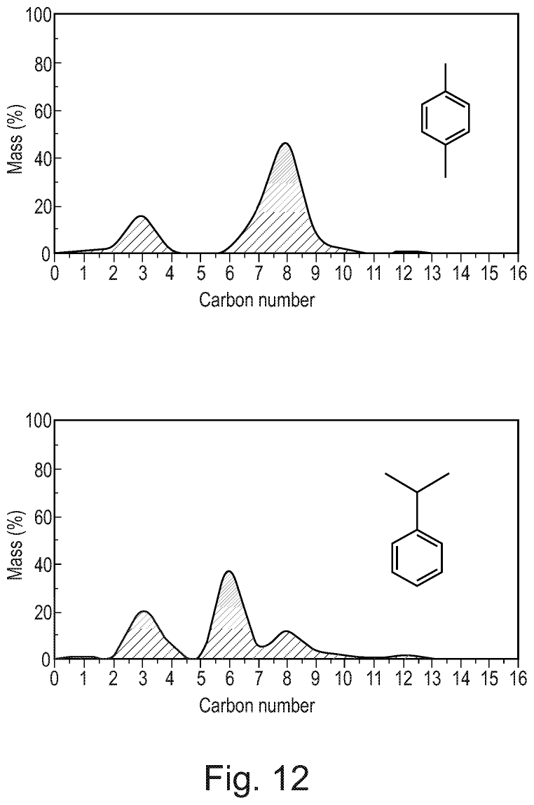

FIG. 12. Product distribution over H-ZSM-5 catalyst with 2 g of cofed xylene under 30 bar of H₂ at 280° C. for 10 h (xylene is included). Product distribution over H-ZSM-5 catalyst with 2 g of cofed cumene under 30 bar of H₂ at 280° C. for 10 h (cumene is included).

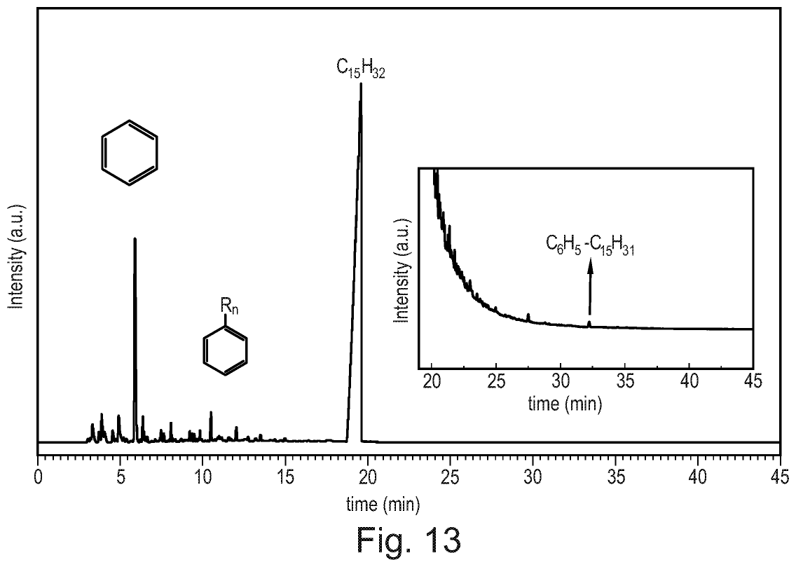

FIG. 13. GC patterns from reaction of C₁₅H₃₂ with 0.2 g of cofeed benzene over H-ZSM-5 catalyst under 30 bar of H₂ at 150° C. for 1 h.

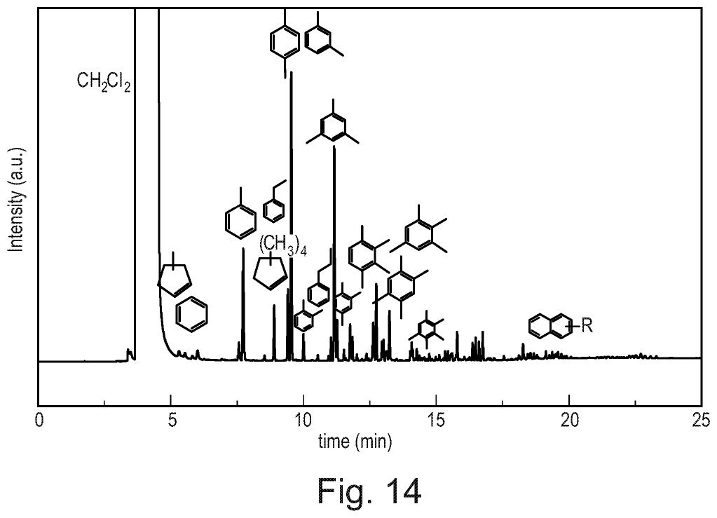

FIG. 14. GC patterns from extracted solution from the solid residue in the spent catalyst of HDPE with 0.2 g of cofed benzene over H-ZSM-5 catalyst under 30 bar of H₂ at 280° C. for 10 h.

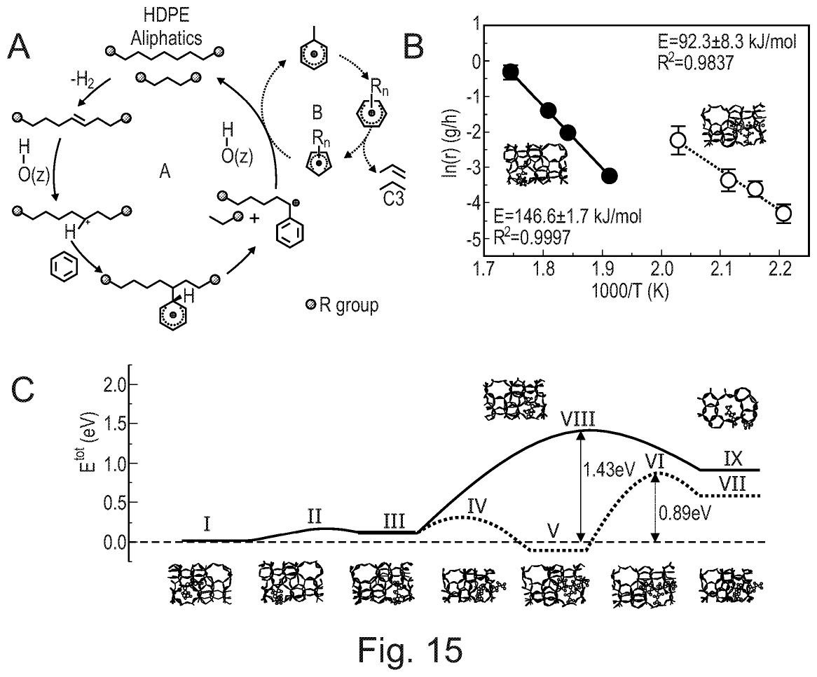

FIG. 15. Mechanistic study of aromatic aided PE decomposition.

FIG. 15A. Paring to form C3 over zeolite via cracking of multi methyl benzene compounds as intermediates in benzene/HDPE reaction mixture.

FIG. 15B. Arrhenius plots of HDPE decomposition over H-ZSM-5 with and without cofed benzene.

FIG. 15C. DFT and Molecular dynamics (MD) calculations of the decomposition of C₈H₁₆ and C₆H₅—C₈H₁₇+.

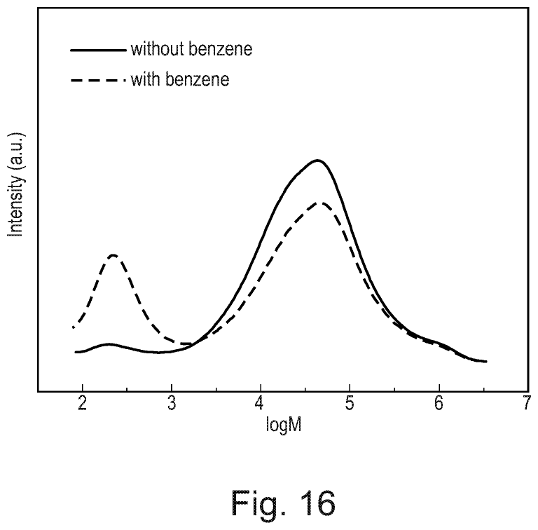

FIG. 16. GPC patterns from the quenched reaction of HDPE with and without 0.2 g of cofeed benzene over H-ZSM-5 catalyst under 30 bar of H₂ at 250° C. for the 1 h. Benzene can assist the degradation of the HDPE to smaller fragments.

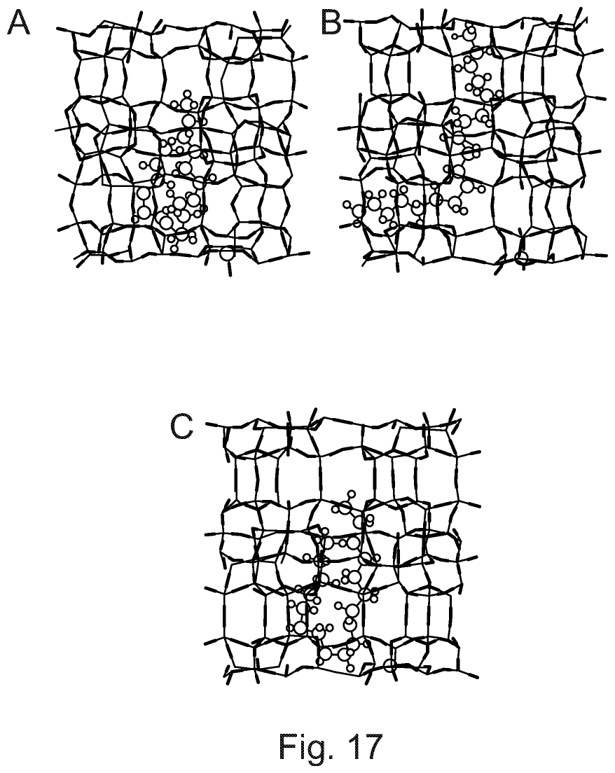

FIG. 17A. The initial status of the globular C₁₈H₃₇+ pin in the H-ZSM-5 pore.

FIG. 17B. The final stretching status of C₁₈H₃₇+ in the H-ZSM-5 pore.

FIG. 17C. MD calculation of stretching of C₁₈H₃₇+ along the H-ZSM-5 pore.

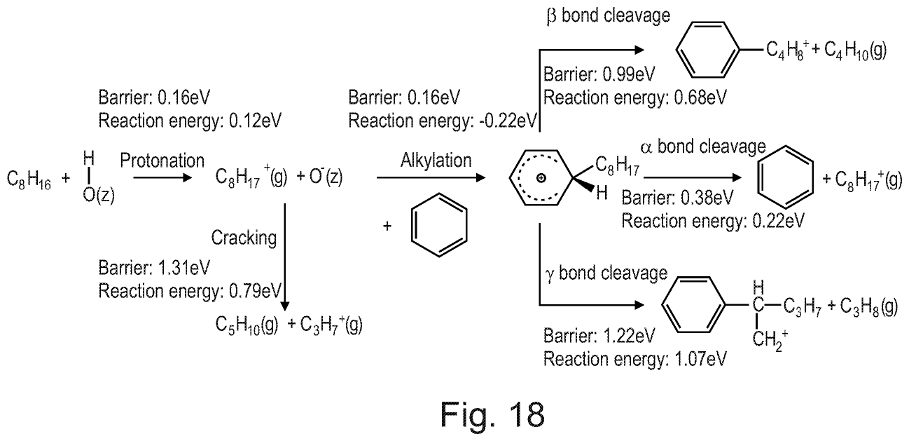

FIG. 18. DFT calculation of cracking C₈H₁₆ with the promotion of benzene over BAS in the H-ZSM-5 pore. β cleavage for both sides of the aromatic ring (1.03 eV barrier) will create methylbenzene (only one cleavage is shown).

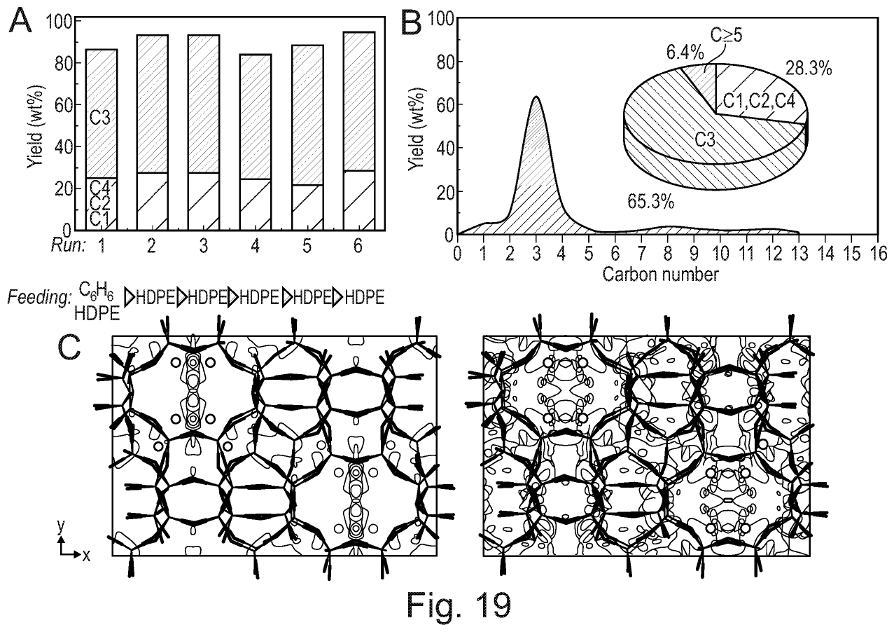

FIG. 19. Recyclability and coke simulations.

FIG. 19A. The yield of gas (C3) in outlet gas for 6 consecutive runs at 280° C. 0.2 g of benzene and 2 g of HDPE were added in the first run, followed by adding 2 g of HDPE each run (10 h) after replacing the produced gas with H₂ (30 bar) for new runs.

FIG. 19B. The product distribution in mass for the 6th reaction run.

FIG. 19C. Coke simulation in the pore of H-ZSM-5 through 250° C., 10 h with (left) and without (right) cofeeding benzene. Only positive carbon density is shown in yellow region. The zeolite framework is shown in red and blue bar.

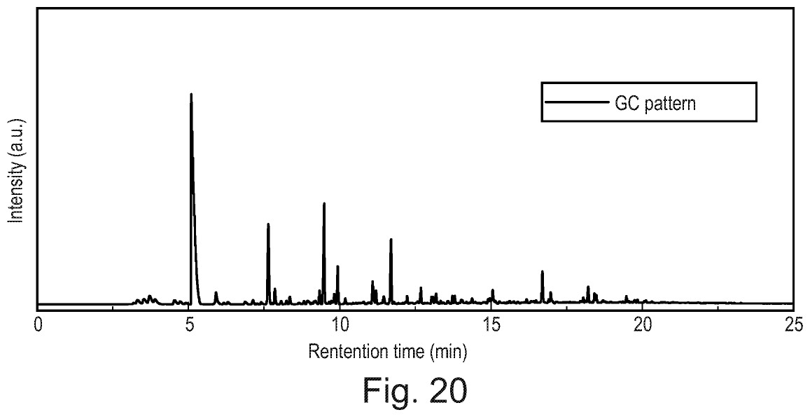

FIG. 20. GC pattern of the liquid product after 6 runs of reaction. Reaction condition: 280° C., 10 h, Benzene, 0.2 g.

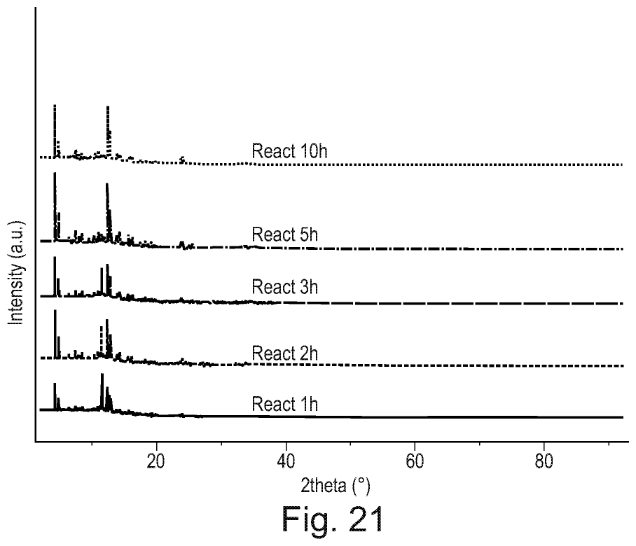

FIG. 21. Synchrotron XRD (SXRD) patterns for the dried mixture of catalyst powder and solid residue after reaction at 280° C. for differing reaction times.

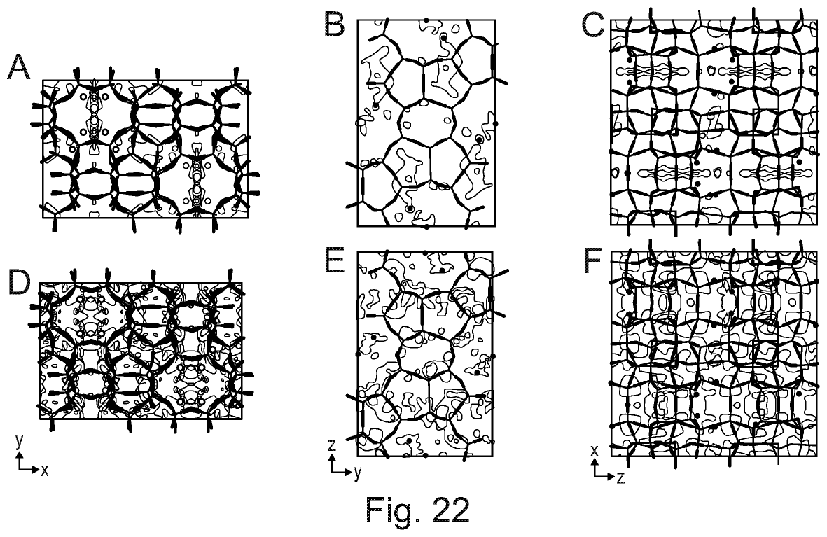

FIG. 22. Coking simulation through sXRD in different direction in H-ZSM-5 for the spent catalyst from HDPE decomposition with the aid of benzene (A, B and C) and without the aid of benzene (D, E and F). Reaction conditions, 250° C., 10 h, 30 bar of H₂.

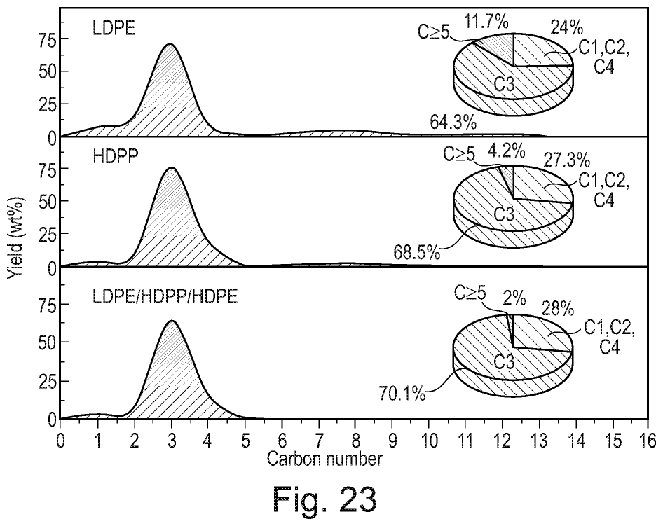

FIG. 23. The product distribution for LDPE and HDPP over H-ZSM-5 with the aid of the benzene initiator. Reaction condition 280° C., 10 h, 30 bar H₂, benzene, 0.2 g.

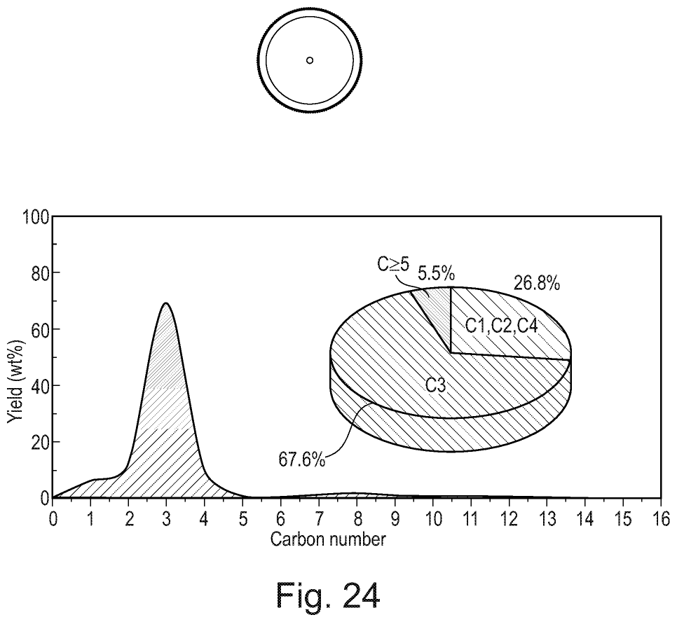

FIG. 24. The product distribution for bottle caps (HDPE) over H-ZSM-5 with the aid of benzene initiator. 280° C., 10 h, 30 bar H₂, benzene, 0.2 g.

### MATERIALS AND METHODS

HDPE (Mw, 88707 g/mol; Mn, 10794 g/mol) were obtained from SCG ltd. LDPE (1546809-3STRIPS) and HDPP (ERMEC591) were purchased from Sigma Aldrich. All the polymers were used without further pre-treatment. Zeolites including H-ZSM-5, H-SAPO-34, and H—Y were obtained from State Key Laboratory of Catalysis, Dalian Institute of Chemical Physics, China. All the zeolites were pre-treated at 550° C. ramping at 5° C. min⁻¹ under a flow of nitrogen (50 mL min-1) for 5 h prior to use. SiO₂Al₂O₃ Grade 135 were ordered from Sigma-Aldrich. Chloroform, dichloromethane (puriss.p.a., reag.ISO, reag.Ph.Eur., 99.0-99.4% GC), Benzene (anhydrous, 99.8%), and Toluene (anhydrous, 99.8%) were purchased from Sigma-Aldrich.

### Characterisation

High-resolution transmission electron microscopy (HRTEM): HRTEM images were obtained by using a JEOL 3000F microscope operated at 300 kV. The samples were prepared by pipetting a drop of the sample dispersion in ethanol onto holey carbon-coated copper mesh grids (400 meshes).

X-ray powder diffraction (XRD): Laboratory XRD was conducted on a Bruker D8 Advance X-ray diffractometer at 40 mA and 40 kV with Cu Kα1 radiation (Δ=1.54056 Å).

¹H and ¹³C Nuclear magnetic resonance (NMR) of products: Prior to the NMR measurement, gas samples were dissolved into CD₂Cl₂ through vacuum transfer on a Schlenk line to a J Young NMR tube. For the liquid, the liquid product collected was added into CD₂Cl₂ and ¹H NMR spectra were recorded at 400 MHz on a HG400 spectrometer. ¹³C NMR were acquired at 400 MHz on a Bruker NEO 400 nanobay. For the solid-state magic angle spinning (MAS) NMR experiments, the samples were first dried in vacuum at 80° C. overnight prior to measurements on a HXY 400 NMR spectrometer. The spectra were analysed using MestReNova x64.

N₂ physisorption: N₂ adsorption isotherm were measured at 77 K and up to 1 bar on a Micromeritics Tristar instrument. All sorption isotherms were collected by using ultrahigh purity N₂ (99.999%). Prior to the sorption measurement, a sample of 0.1-0.2 g was added into a sample tube subjected to a vacuum of 10-5 Torr at 673 K for 12 h. The measurements were carried out by N₂ at 77 K. N₂ desorption data with an initial slope (0.01 to 0.1 P/P₀) permitted calculation of the apparent surface areas based on the BET equations. t-plot method was used to calculate the external and internal surface area.

Gel permeation chromatography (GPC): GPC was performed on a high temperature gel permeation chromatograph with an IR5 infrared detector (GPC-IR5). Samples were prepared by dissolution in 1,2,4-trichlorobenzene (TCB) containing 300 ppm of 3,5-di-tert-buty-4-hydroxytoluene (BHT) at 160° C. for 90 minutes and then filtered with a 10 μm SS filter before being passed through the GPC column. The samples were run under a flow rate of 0.5 mL min-1 using TCB containing 300 ppm of BHT as mobile phase with 1 mg mL-1 BHT added as a flow rate marker. The GPC column and detector temperature were set at 145° C. and 160° C. respectively.

Coke simulation from synchrotron powder X-ray diffraction (SXRD): SXRD were conducted on Beamline 111, 9 Diamond Light Source, UK. The energy of the incident X-ray flux was set at 15 keV. The wavelength and the 20-zero point were refined using a diffraction pattern obtained from a high-quality silicon powder (SRM640c). The samples were calcined at 600° C. under N₂ for 6 h to remove all the other residues except the coke. The obtained sample powder was then ground and loaded into a 0.5 mm borosilicate glass capillary. High-resolution SXRD data of all samples were achieved by using the multi-analyzer crystals (MAC) detectors in the 20 range 0-150° with 0.001° data binning. Each MAC pattern was collected for 1 h to get satisfying statistics. Rietveld fitting were performed with 4 millidegrees (mdeg) resolution SXPD data. 8 carbon sites for each pattern were chosen to simulate the diffuse electron density for coke. Global thermal parameters for oxygen and tetrahedral sites (Si/Al) were allowed to refine against all scans. Pure H-ZSM-5 is used as background for the series of patterns in order to fit the difference between patterns using the 8 carbon sites. However, the diffuse electron density is not limited in the 8 sites based on coordination and occupancy refinement, allowing refinement within the pore to model excess/unmodelled density in the pore. The thermal parameters were refined but always provided a maximum value of 20 for both solvent background and carbon, indicating the diffuse electron density. F_{obs}−F_{calc} mode were adopted to the simulation with 8 individual carbons, but also includes 4 background solvent containment. Only positive density is shown with electron Isosurface=0.12. The occupancy unit is the summed occupancy of the 8 asymmetric carbon sites which can be larger than 8.

### Catalyst Test

The catalysis was carried out in a 50 mL autoclave, where 2 g of polymer was charged. In most cases, 0.2 g of catalyst and 0.2 g of aromatic compound were also added. The autoclave was evacuated to remove air and then filled with hydrogen gas at a pressure of 30 bar unless otherwise specified. The temperature was increased to the target temperature in 1 h. The reactor was kept at constant stirring with a glassy coated stirrer. The autoclave was allowed to cool to room temperature before analysis. The gaseous product was analysed by gas chromatography (GC) and the liquid phase product was analysed by GC mass spectroscopy (Agilent GC-MS 6890). The liquid product was collected through centrifugation prior to injecting into the GC-MS in order to separate solid from the mixture. In some cases, the liquid product had to be diluted with chloroform or dichloromethane in order to get sufficient liquid to analyse from the catalyst, and to ensure that all of the liquid products had been washed off and collected from the interior surface of the reactor autoclave. The solid from the centrifugation was then dried in a vacuum oven at 80° C. overnight and weighted. The net weight of solid residue was obtained by taking the weight of catalyst into account.

After the reaction, products were formed of three phases, namely gas, liquid, and solid. The weight of autoclave including the stirrer was first taken with a range and accuracy of 5 kg±0.05 g. The gas mass was determined by the weight difference of the autoclave before and after releasing the gas, taking the weight of H₂ or N₂ into account. The liquid mass was obtained from the weight difference of the autoclave after discharging the gas and emptying the autoclave (stirrer included).

The mass yield (Yieldᵢ, wt %) of each species except arenes were obtained through the following equation:

$${Yield}_{i} ( wt \% ) = ( {m}_{i} ) / {m}_{P E} \qquad ( 1 )$$

where mᵢ is the weight of the corresponding species, is the mass of PE initially added.
The mass yield of arene

$${Yield}_{Arene} ( wt \% ) = ( {m}_{Arene} - {m}_{Benzene} ) / {m}_{P E} \qquad ( 2 )$$

where m_{Arene} is the weight of the arene, m_{Benzene} and m_{PE} are the mass of benzene and PE initially added.
The mole yield (% C) and balance were calculated based on the following equations:

$${Yield}_{Arene} ( \% C ) = ( {M}_{Arene} - {M}_{Benzene} ) / {M}_{PE} \qquad ( 3 )$$

where M_{Arene} is the carbon amount in arenes based on molar amount, M_{Benzene} is the carbon amount in benzene initially added based on molar amount, M_{PE} is the carbon amount in polymer initially added based on molar amount.

$${Yield}_{gas} ( \% C ) = {M}_{g a s} / {M}_{P E} \qquad ( 4 )$$

where M_{gas} is the carbon amount in C1-C4 species based on molar amount.

$${Yield}_{C 3} ( \% C ) = {M}_{C 3} / {M}_{P E} \qquad ( 5 )$$

where M_{C3} is the carbon amount in C3 based on molar amount.

$$Balance = ( M - {M}_{Benzene} ) / {M}_{P E} \qquad ( 6 )$$

where M is the carbon amount in products including gas, liquid and solid residue based on molar amount.

In order to release the products trapped in zeolite pores, the dried spent catalyst was digested by 50 vol % hydrofluoric acid (HF) solution at room temperature for 30 min. HF was neutralized by using a NaOH solution. The obtained solution was extracted by CH₂Cl₂ and then analysed by GCMS.

### Results and Discussion

C3 Production from Benzene Promoted HDPE/PP Decomposition

In the course of searching for a new method for HDPE activation, a tiny amount of benzene (228 μL, 0.2 g) was added to 2 g of HDPE with 0.2 g of H-ZSM-5. After reaction at 280° C. for 10 h under 30 bar of H₂, all the added HDPE powders were completely converted to gaseous and liquid products under such a mild condition even without using transition metal catalysts to promote the conversion. In general, noble metals such as Pt or Ru are used to promote the HDPE decomposition and allow the reaction to proceed at or lower than temperatures of 300° C.^{6,16,17}. Advantageously, in the present invention, most of the added HDPE was converted to gaseous products (91 wt %, C1-C4) with a small amount of liquid obtained over the H-ZSM-5 without any metal inclusion. C3 (96% propane and 4% propylene) species are the main gaseous product with a yield up to 75 wt %. This selective cleavage of polyolefins to monomer-like fragments at mild conditions is particularly interesting given the strong C—C bonds present in PE and PP. It is well known that propane, which is the main component formed, can be easily dehydrogenated to propene and/or cracked to ethylene, indicating that the method of the present invention can be used for chemical recycling of thermoplastic polymers into useful products.

In the present invention, C3 products are formed in the gas phase from PE/PP thereby introducing a new closed cyclic loop for plastics under mild conditions. The reaction proceeds over commercial HZSM-5, which has a typical MFI structure with straight 10-ring channels running parallel to the corrugations and sinusoidal 10-ring channels perpendicular to the nano sheets (FIG. 1A). The size of the pore window is around 5.4-5.6 Å. Raw HDPE shows characteristic peaks at 21.2° and 23.7°, which correspond to (110) and (200). These peaks completely disappear after the reaction while the characteristic peaks of H-ZSM-5 are retained, as shown in FIG. 1B, indicating that the entire polyolefin is decomposed while the zeolite remains. FIGS. 1C and 1D show the product distribution and mass yields for HDPE decomposition over H-ZSM-5 with and without cofeeding benzene. Without benzene addition, there is around 40% of HDPE residue remaining for traditional HDPE decomposition at 280° C. for 10 h and a wide C range of products is obtained in the liquid and gas products. In contrast, no solid residue from HDPE remains after a tiny amount of benzene addition at the initial state of the reaction. In the presence of benzene, the conversion rate is significantly improved with C3 formation in the gas phase. The benzene promoted PP decomposition was also investigated and showed very similar activity and product yield.

### Effect of Benzene on HDPE Decomposition

To study the effect of benzene, a milder temperature of 250° C. was adopted. It was found that the addition of benzene into the reaction could greatly improve the rate of C—C bond cleavage in the HDPE over H-ZSM-5 catalyst under H₂ at 250° C. The liquid product was separated from the solid catalyst and plastic residue mixture by centrifugation (FIG. 2A). The liquid product appeared to be orange in colour, while the catalyst and plastic residue were collected in the bottom of the centrifuge tube. The weights of all the products were measured (FIG. 2B). Without benzene, there is only a small degree of HDPE conversion to liquid products of a broad range of products (heavy hydrocarbons), presumably via cracking of —CH₂CH₂— moiety in contact with BAS of the zeolite²¹⁻²³.

Interestingly, HDPE degradation rates are significantly enhanced by the addition of benzene. Increasing the amount of benzene further decreases the amount of plastic residue and raises the yield of liquid product. When the benzene amount is raised to 1.0 g (76.9 mmol C), more than 90% of the 2.0 g (142.9 mmol C) of HDPE can be converted within 1 h. Based on the mass values, the rate of HDPE conversion is progressively boosted from 0.19 g/h to 1.99 g/h, as shown in FIG. 2C when increasing the amount of benzene at 250° C. The liquid product was analysed by GC-MS while the gas product was determined by GC. The mass balance was generally found to be higher than 90% (FIG. 2B).

The reaction temperature was increased to 280° C. with 0.2 g of benzene and 2 g of HDPE over 0.2 g of H-ZSM-5. The product distribution on mass basis (benzene is included) is shown in FIG. 2D. After 0.5 h, over 90% of HDPE had been converted and the major products are still in the form of liquid arenes with carbon number ≥5. However, longer reaction times enhanced the conversion of HDPE with full decomposition after 5 h, the major products being gaseous species (75% C3 in 10 h), as seen from FIG. 2D. The GC patterns are shown in FIG. 3. It is interesting to find that the predominant products are multi substituted methyl-benzene compounds (arenes) from benzene reacting with the HDPE plastic. There are only trace amounts of benzene left. The formation of substituted methylbenzene compounds indicates that the benzene initiator can scavenge the CH₂ moieties from the polyolefin via benzene alkylation to the alkene position of the polyolefin chain over BAS at elevated temperatures. The removal of an electron from this benzene anchored polyolefin over BAS could lead to the formation of a radical cation, a species capable of undergoing the cleavage of B bonds to the aromatic ring²⁴ from both sides to induce fragmentation of the polymer. β bond cleavage is known to be an essential step in the side-chain oxidation of alkyl aromatics and related compounds²⁴⁻²⁶. Although a substantial amount of arene products may be derived from the reaction of benzene/HDPE, the higher C content of HDPE over benzene clearly indicates the catalytic formation of arenes from the polymer directly according to the reaction stoichiometry. Interestingly, the majority of gas components are C3 in nature (up to 75 wt % in gas) when elevating the reaction temperature and reaction time.

The product distribution in terms of molar amount of carbon conversion (excluding carbon from the benzene promoter) from HDPE was derived. As shown in FIG. 2E and Table 1 (below), the carbon amount in arenes (discounting the carbon from benzene) can reach 53.1% with 20.4% gas and 26.5% C≥5 non-arene hydrocarbons and solid residue from the initial 0.5 h in a reactor. As the reaction time progresses, C1 to C4 gas fractions, particularly C3 gas fractions (propane as the main component, >99%), dramatically increases at the expense of arenes and C≥5 non-arene hydrocarbons. The carbon yield of C3 went up from 11.5% to 49.8% at 280° C., while the carbon yield of arenes simultaneously decreased from 53.1% to 5.6% with no plastic residue after 5 h reaction time. Prolonging the reaction time to 10 h also increased the C3 yield to 70.3% at the expense of other C25 non-arene hydrocarbons (2.8%). Interestingly, C3 is the final product with high selectivity from the controlled degradation of HDPE/arenes/higher hydrocarbons over the H-ZSM-5 as the reaction time increases. Polyolefins, including PE/PP, are currently made from naphtha cracking to an initial propylene/ethylene mixture (higher ratios at low temperatures), followed by their separation and polymerization. Thus, propane dehydrogenation can produce propylene monomers with excellent selectivity for PP. C3 cracking to ethylene suitable for PE may also be possible. Therefore, high C3 selectivity generates new potential routes for a genuine circular economy for thermal polymer recycling. Attempts to run at higher temperatures (>500° C.) or in the presence of a solely N₂ atmosphere to make the propylene directly were not successful as significant solid residues were always found indicating that a H₂ atmosphere with relatively low temperatures are essential. It is theorised that these conditions prevent deactivation of the zeolite.

TABLE 1
Product distribution, yield and carbon balance in molar amounts of carbon at 280° C., 30 bar H₂

| | Substrate | | Product (mmol C) | | | Yield (% C) | | | |
|---|---|---|---|---|---|---|---|---|---|
| | (mmol C) | | Arenes | Non-arene | | Arenes | | | |
| Time | Benzene | | (Ben | Hydrocarbons | Solid | (Ben | Gas^{a} | | Balance |
| (h) | (Ben) | HDPE | incl.) | (gas incl.) | residue | excl.) | (C3) | Others^{b} | (% C) |
| 0.5 | 15 | 143 | 91 | 52 | 15 | 53.1 | 20.4 (11.5) | 26.5 | 100.0 |
| 1 | 15 | 143 | 76 | 72 | 7 | 42.7 | 32.3 (20.9) | 22.9 | 97.9 |
| 5 | 15 | 143 | 23 | 129 | 0 | 5.6 | 70.9 (49.8) | 19.3 | 95.8 |
| 10 | 15 | 143 | 23 | 122 | 0 | 5.6 | 91.0 (70.3) | 2.8 | 99.4 |

^{a}C1-C4; ^{b}C ≥ 5 Non-arene hydrocarbons and solid residue from HDPE.

To prove the importance of molecular accessibility to the BAS sites in the pore of H-ZSM-5³⁰, controlled experiments using zeolites with different pore sizes were conducted. H-ZSM-5 has the lowest surface area compared with SAPO-34 and H—Y as shown in FIG. 4A and Table 2. Interestingly, as seen from Table 3, the same amount of benzene (arene) is retained with almost no other arenes produced in the final product when H-SAPO-34 is used (pore window of 3.8 Å does not allow benzene to gain access to the BAS). Approximately 50.0% of the HDPE is retained in the solid residue with the rest being converted into a range of heavy non-arenes as cracking products. This indicates that the slow HDPE cracking does not benefit from benzene addition in H-SAPO-34 (FIG. 4B). On the other hand, when the H—Y zeolite (pore size 7.5 Å and a pore window of 10.0 Å) is investigated, the conversion of HDPE is significantly higher than that over H-SAPO-34. The predominant products, however, are arenes instead of C3 species. This indicates that the pore dimension and geometry of the zeolite used is important for C3 product selectivity. It is theorised that the pore structure in H-ZSM-5 enhances the formation of C3 products via the cracking of trapped methylbenzene compounds^{29,31-33}. H—Y, which has much larger pore size, is not the right pore structure to operate via this mechanism. Further controlled benzene promoted experiments using acidic SiO₂Al₂O₃ under N₂ indicated that the BAS sites act as the active sites for the reaction (FIG. 5).

To confirm the important role of Bronsted acid sites (BAS: H—O(z)) in this reaction, the acidity of H-ZSM-5 was varied (FIG. 4D). Interestingly, a linear relationship between the HDPE conversion and increasing BAS acidity (decreasing SiO₂/Al₂O₃ ratios) is shown, indicating that the BAS is the active site for this reaction.

TABLE 2
Surface area obtained from N₂ sorption

| | BET | Micropore Area | t-Plot External Surface Area |
|---|---|---|---|
| Zeolite | (m²/g) | (m²/g) | (m²/g) |
| H-SAPO-34 | 487.4 | 475.4 | 12.0 |
| H-ZSM-5 | 303.1 | 210.8 | 92.3 |
| H-Y-15 | 612.7 | 527.0 | 85.7 |

Micropore area and external surface area are obtained through t-Plot method.

TABLE 3
Product distribution, yield and carbon balance in molar amounts of carbon at 280° C., 30 bar H₂ for 10 h.

| | Substrate | | Product (mmol C) | | | Yield (% C) | | | |
|---|---|---|---|---|---|---|---|---|---|
| | (mmol C) | | Arenes | Non-arene | | Arenes | | | |
| | Benzene | | (Ben | Hydrocarbons | Solid | (Ben | Gas^{a} | | Balance |
| Cat. | (Ben) | HDPE | incl.) | (gas incl.) | residue | excl.) | (C3) | Others^{b} | (% C) |
| HSAPO-34 | 15 | 143 | 15 | 70 | 83 | 0.0 | 4.6 (1.6) | 101.7 | 106.3 |
| H-Y | 15 | 143 | 84 | 63 | 16 | 49.3 | 13.9 (3.4) | 39.3 | 103.1 |

^{a}C1-C4; ^{b}C ≥ 5 Non-arene hydrocarbons and solid residue.

Interestingly, using hexane as an initiator did not show any clear improvement in the rate of HDPE cleavage to the desired products, highlighting the importance of aromatics to aid the selective conversion of polyolefins to C3 species. This is confirmed in FIG. 6, whereby promotion with alkyl aromatics such as toluene and xylene showed a similar product distribution to that of benzene promotion. This indicates that aromatic compounds other than benzene are suitable for the selective production of C3 species in the catalytic cycle.

To track the fate of benzene, unlabelled or isotopic ¹³C labelled benzene was cofed to promote the HDPE decomposition and the gaseous, liquid and solid reaction products were monitored by ¹H and ¹³C NMR. As shown in FIG. 7A, methylene and methyl groups in the gas products are observed with an average ratio of 0.36, similar to that of propane (Table 4), which is identified by the GC as the main product.

TABLE 4
Molar ratio of H in methylene group to H in methyl group for C1-C4

| | CH₄ | CH₃CH₃ | CH₃CH₂CH₃ | CH₃CH₂CH₂CH₃ |
|---|---|---|---|---|
| H_{—CH2—}/H_{—CH3} | — | 0 | 0.33 | 0.67 |

| | CH₂═CH₂ | CH₂═CHCH₃ | CH₃CH═CHCH₃ | CH₂═CHCH₂CH₃ |
|---|---|---|---|---|
| H_{—CH2—}/H_{—CH3} | — | 0.67 | — | 1.33 |

When ¹³C benzene was used, new splitting peaks for ¹³C labelled methyl and methylene groups arose, indicating that the added ¹³C benzene can be converted to propane (FIG. 7D). In the liquid phase, the number of ¹H in the aromatic compounds when compared to the total H number (H_{ar+α}/Hₜₒₜₐₗ) is 0.66 (FIG. 7B), indicating that the predominant species are aromatic compounds. H_{-Ph-CH3}/H_{α}=0.77 further suggests that the main species are methyl-substituted benzenes as indicated from GCMS instead of polyaromatics. For isotopic labelled benzene, ¹H NMR signal splitting was observed due to ¹³C-¹H coupling. However, a sharp peak is also observed at around 7.5 ppm (aromatic region), which was not split by ¹³C (FIG. 7E) in the liquid product from the cofed of ¹³C benzene and HDPE, indicating a substantial part of aromatics in the liquid are derived from the HDPE. ¹H ssNMR for the solid residue showed some alkylbenzenes were retained in the dried sample (FIGS. 7C and 7F).

Interestingly the ¹³C signal is significantly enhanced when ¹³C benzene are cofed (FIG. 8). In the gas phase, both the aromatics and aliphatic (C1-C4) show more intense ¹³C signals compared to products obtained from the experiments with non-labelled benzene with the same number of scans, indicating that ¹³C benzene can be converted into gas products. However, in the liquid phase, only the aromatics signals are notably enhanced, demonstrating that the benzene may not contribute much to form the aliphatic liquid (C≥5). When the reaction is carried out without benzene, the aromatic content is less as indicated by the lower ¹H and ¹³C signals (FIG. 9). Similar changes to the NMR signal are also observed with the reaction is operated at 250° C. for 1 h (FIGS. 10 and 11). From the NMR measurements, it can be deduced that initial added benzene can be converted into alkylbenzenes, and then to C3 species rather than to aliphatic hydrocarbons (C≥5).

To further confirm this, controlled experiments using 2 g of benzene, xylene, and cumene without HDPE were conducted. It was found that benzene cannot be reacted as there was no gas and liquid product detected. On the contrary, xylene and cumene can be converted into C3 as the main gas product (FIG. 12). This supports the theory that C3 gas is mainly derived from the methylbenzene compounds but not from benzene^{29,31-33}. The reaction between benzene and HDPE was simulated by replacing the HDPE with a model compound of C₁₅H₃₂. Interestingly, C₆H₅—C₁₅H₃₁ is detected from the GCMS (FIG. 13), indicating that alkylation of benzene can take place under these conditions.

### Hydrocarbon Pool Mechanism Induced by Added Aromatics

To investigate the organic intermediates prior to C3 species production in the pores of the H-ZSM-5 catalyst, HF solution was used to dissolve the dried catalyst and the hydrocarbons inside were then extracted with dichloromethane. The GC pattern of the extracted liquid products (FIG. 14) shows that the majority of species are methylbenzene and multi-substituted methyl-benzene compounds with a small amount of methyl cyclopentene and unused benzene. It is known that in the methanol to hydrocarbon (MTH) process, benzene can be initially formed by a carbon source (i.e., methanol dehydrogenation) on the BAS at elevated temperatures in the zeolite channels due to template aromatization. Their further carbon capture from methanol on BAS can form multi-substituted methyl-benzene compounds. Upon cracking, these organic species will lead to selective C3 formation via the ‘hydrocarbon pool mechanism’^{27,28,29}. In such process, substituted methylbenzene species are the key intermediates, which are also identified in our liquid products, as shown in FIG. 15A.

It is theorised that similar ‘hydrocarbon intermediates’ are created in H-ZSM-5 from the benzene/polyolefin mixture to form gaseous products, in particular C3 species, under our low temperature conditions. Gel permeation chromatography (GPC) was also employed to measure the molecular weight distribution of solid residue from the quenched reaction at 250° C. for 1 h. As shown in FIG. 16, with cofeeding benzene the broad peak at the lower molecular weight significantly increases, whereas the higher molecular weight peak decreases. This confirms the promoting effect of benzene on the decomposition of HDPE to lower molecular mass products.

The mechanism of aromatic aided HDPE decomposition is proposed in FIG. 15A: at least two reaction cycles are envisaged according to the ‘hydrocarbon pool mechanism’, leading to the decomposition of the polymer (1). The HDPE hair-pin is dehydrogenated to corresponding alkene and protonated as carbocation in the pore of H-ZSM-5. Alkylation then takes place between the carbocation and benzene. Selective β-bond cleavage of the obtained alkylbenzene results in decomposition of the HDPE chain to shorter aliphatic hydrocarbons and methylbenzene compounds. The introduction of benzene clearly induces the alkylation and the production of methylbenzene compounds (2). These methylbenzene compounds make up the ‘hydrocarbon pool’ in the pores of the HZSM-5 in a similar manner to the MTH process, resulting in production of the C3 compounds upon thermal cracking in autocatalytic manner. It is noted propylene is the major product if the reaction is run in a N₂ atmosphere (similar to the MTH process). However, the presence of H₂ in the atmosphere proved essential to avoid a rapid deactivation of the catalyst. Hydrogenation of the in-situ propylene by H-ZMS-5, leading to the formation of propane, was confirmed by feeding 1.5% C₃H₆/98.5% H₂, 30 bar, 280° C. 10 h over pure H-ZSM-5. In this investigation, 95% of the propylene was converted to propane.

DFT and MD calculations were employed to monitor the HDPE cracking on BAS with and without cofed benzene in circle 1 of FIG. 15A. As shown in FIG. 17, the hairpin of folded C₁₈H₃₈ chain (to model HDPE) could be stretched into linear chain moieties in the pore of H-ZSM-5 by the wall effect. To simplify the modelling, the shorter C₈H₁₆ was chosen as the hydrocarbon model for decomposition to reduce interaction with BAS in other units (FIG. 18). Arrhenius plots of HDPE decomposition over H-ZSM-5 indicates that cofed benzene can reduce the apparent energy barrier from 146.6 to 92.3 KJ/mol for HDPE decomposition on the active site (FIG. 15B). On a BAS site, C₈H₁₆ is protonated to C₈H₁₇⁺ with an energy barrier of 0.40 eV (FIG. 15C). The carbocation then reacts with benzene to form methylbenzene with an energy barrier as low as 0.16 eV. The rate limiting step to decompose C₈H₁₆ is cracking of the energetic C—C into smaller hydrocarbon fragments without benzene (˜2 eV). For the cofed benzene, a much lower energy barrier is obtained. The overall energy barrier for the benzene aided C₈H₁₆ cracking is roughly 0.89 eV (around 85.4 KJ/mol), which is consistent with the apparent energy barrier (92.3 KJ/mol) obtained from the experimental Arrhenius plot (FIG. 15B). The calculations (FIGS. 15C and 18) also provide support for the β bond cleavage to produce methylbenzene. Thus, this investigation indicates that the C moieties in HDPE are functionalised by benzene to form methylbenzene and trigger the hydrocarbon pool autocatalysis to form C3 species.

### Recyclability of Thermal Plastics to C3 Molecules

Since aromatic compounds such as benzene, toluene and substituted methylbenzene compounds of the ‘hydrocarbon pool’ intermediates may enter to the catalytic cycle to capture carbon from HDPE and form C3 species, fresh HDPE was added to test the recyclability of the present invention without adding additional aromatic promoter. The addition of the fresh HDPE was made into the same wet catalyst mixture after opening the reactor (liquid product with H-ZSM-5) for each run (10 h) in a total of 6 runs after replacing the gas product with H₂. Consistently, a yield in excess of 60% was obtained for the C3 species (FIG. 19A, Table 5) for each run with reference to the carbon content of the fresh HDPE added in the 6 batches. A linearly increase in accumulating yield from 132 mmol (1^{st} run) to 787 mmol (6^{th} run—no analysis for liquids except the final 6th run) was shown. The final accumulated liquid content was higher than the first run, which was collected and analysed by GCMS (FIG. 20), confirming the presence of substituted methylbenzene compounds. The complete product distribution for the 6 runs is shown in FIG. 19B. This suggests that the zeolite is not deactivated and can be totally recycled without further addition of benzene or toluene initiator, but instead using the liquid by-products in the catalytic cycle. Thus, the present invention is of practical importance in considering of the cost of initiator and catalyst stability in this synthetic method.

TABLE 5
Product distribution, yield and carbon balance in carbon mole at 280° C., 30 bar H₂ (10 h each)

| | Substrate | | Product (mmol C) | | | Yield (% C) | | | |
|---|---|---|---|---|---|---|---|---|---|
| | (mmol C) | | Arenes | Non-arene | | Arene | | | |
| Entry | Benzene | | (Ben | Hydrocarbons | Solid | (Ben | Gas^{a} | | Balance |
| (run) | (Ben) | HDPE | incl.) | (gas incl.) | residue | excl.) | (C3) | Others^{b} | (% C) |
| 1 | 15 | 143 | — | 132 | — | — | 92.4 (71.3) | — | — |
| 2 | — | 143 | — | 134 | — | — | 93.4 (66.3) | — | — |
| 3 | — | 143 | — | 131 | — | — | 91.9 (68.8) | — | — |
| 4 | — | 143 | — | 128 | — | — | 89.7 (69.2) | — | — |
| 5 | — | 143 | — | 126 | — | — | 88.3 (66.6) | — | — |
| 6 | — | 143 | — | 146 | — | — | 95.0 (66.4) | — | — |
| Sum^{c} | 15 | 858 | 27 | 787 | 0 | 1.4 | 91.7 (68.1) | 1.3 | 94.4 |

^{a}C1-C4 ^{b}C ≥ 5 Non-arene hydrocarbons and solid residue; cumulative sum for the 6 runs of reaction.

Coke deposition leading to catalyst deactivation is a known issue for MTH reactions. The sXRD for the spent catalyst was undertaken (FIG. 21) and the carbon density of coke in the voids was simulated. As shown in FIGS. 19C and 22, it is interesting to find that with benzene addition, the coke density in the pore of H-ZSM-5 is significantly lower than that without the benzene in a H₂ atmosphere. The occupancy for the spent catalyst after the reaction without benzene is 2.6 times higher than that with cofed benzene (7.33C vs. 2.79C per unit cell) according to refinement. This suggests that the carbon scavenging ability of benzene under a H₂ atmosphere can also reduce the coke precursors to be built up in the zeolite.

### Nature of Thermoplastics

The selective conversion of thermal plastics to C3 molecules was further investigated with LDPE, HDPP and their mixture with HDPE. As shown in FIG. 23 and Table 6, conversion of LDPE and HDPP was also be enhanced with the addition of benzene to give high selectivity to the C3 species. The yield of C3 is 68.8% for HDPP. Similar yield can be obtained over commercial HDPE bottle caps (FIG. 24). Thus, the regeneration of non-biodegradable and chemically stable PP to C3 monomers in a circular economy of this thermal plastic recycling is made possible by the present invention.

TABLE 6
Product distribution, yield and carbon balance in carbon mole at 280° C., 30 bar H₂ for 10 h.

| | Substrate | | Product (mmol C) | | | Yield (% C) | | | |
|---|---|---|---|---|---|---|---|---|---|
| | (mmol C) | | Arenes | Non-arene | | Arene | | | |
| | Benzene | PE/ | (Ben | Hydrocarbon | Solid | (Ben | Gas^{a} | | Balance |
| Polymer | (Ben) | PP | incl.) | (Gas incl.) | residue | excl.) | (C3) | Other ^{b} | (% C) |
| LDPE | 15 | 143 | 30 | 132 | 0 | 10.1 | 88.2 (81.9) | 4.0 | 102.3 |
| HDPP | 15 | 143 | 19 | 140 | 0 | 2.5 | 96.2 (88.8) | 1.6 | 100.3 |
| LDPEHDP | 15 | 143 | 10 | 135 | 0 | 0.4 | 88.2 (63.2) | 7.3 | 95.9 |
| PHDPE | | | | | | | | | |

^{a}C1-C4; ^{b} C ≥ 5 Non-arene hydrocarbons and solid residue.

While specific embodiments of the invention have been described herein for the purpose of reference and illustration, various modifications will be apparent to a person skilled in the art without departing from the scope of the invention as defined by the appended claims.

### REFERENCES

1 Adyel, T. M. Accumulation of plastic waste during COVID-19. Science 369, 1314-1315 (2020).
2 Vollmer, I., Jenks, M. J., González, R. M., Meirer, F. & Weckhuysen, B. M. Plastic waste conversion over a refinery waste catalyst. Angew. Chem. 133, 16237-16244(2021).
3 Benson, N. U., Bassey, D. E. & Palanisami, T. COVID pollution: impact of COVID-19 pandemic on global plastic waste footprint. Heliyon 7, e06343(2021).
4 Jehanno, C. et al. Selective chemical upcycling of mixed plastics guided by a thermally stable organocatalyst. Angew. Chem. Int. Ed. 60, 6710-6717(2021).
5 Plastic upcycling. Nature Catal. 2, 945-946(2019).
6 Zhang, F. et al. Polyethylene upcycling to long-chain alkylaromatics by tandem hydrogenolysis/aromatization. Science 370, 437-441(2020).
7 Tennakoon, A. et al. Catalytic upcycling of high-density polyethylene via a processive mechanism. Nature Catal. 3, 893-901(2020).
8. Aisien, E. T., Otuya, I. C., Aisien, F. A. Thermal and catalytic pyrolysis of waste polypropylene plastic using spent FCC catalyst. Environ. Technol. Innov. 22, 101455(2021).
9. Palos, R., Gutierrez, A., Vela, F. J., Olazar, M., Arandes, J. M., Bilbao, J. Waste Refinery: The Valorization of Waste Plastics and End-of-Life Tires in Refinery Units. A Review. Energy Fuel 35(5), 3529-3557(2021).
10 Jie, X. et al. Microwave-initiated catalytic deconstruction of plastic waste into hydrogen and high-value carbons. Nature Catal. 3, 902-912(2020).
11 Tournier, V. et al. An engineered PET depolymerase to break down and recycle plastic bottles. Nature 580, 216-219(2020).
12 Wei, R. et al. Possibilities and limitations of biotechnological plastic degradation and recycling. Nature Catal. 3, 867-871(2020).
13. Hong, M., Tang, X., Newell, B. S., Chen, E. Y. X. “Nonstrained” γ-butyrolactone-based copolyesters: copolymerization characteristics and composition-dependent (thermal, eutectic, cocrystallization, and degradation) properties. Macromolecules, 50(21), 8469-8479(2017).
14. Zumstein, M. T., Schintlmeister, A., Nelson, T. F., Baumgartner, R., Woebken, D., Wagner, M., Kohler, H. P., McNeill, K., Sander, M. Biodegradation of synthetic polymers in soils: Tracking carbon into CO₂ and microbial biomass. Sci. Adv 4(7), eaas9024(2018).
15. Richert, A., Dąbrowska, G. B. Enzymatic degradation and biofilm formation during biodegradation of polylactide and polycaprolactone polymers in various environments. Int. J. Biol. Macromol 176, 226-232(2021).
16 Lu, S. et al. H₂-free plastic conversion: Converting PET back to BTX by unlocking hidden hydrogen. ChemSusChem (2021). Doi.org/10.1002/cssc.202100196.
17 Jing, Y. et al. Towards the circular economy: Converting aromatic plastic waste back to arenes over a Ru/Nb205 catalyst. Angew. Chem. Int. Ed. 133, 5587-5595(2021).
18. Vollmer, I., Jenks, M. J., Roelands, M. C., White, R. J., van Harmelen, T., de Wild, P., van der Iaan, G. P., Meirer, F., Keurentjes, J. T. F., Weckhuysen, B. M. Beyond mechanical recycling: Giving new life to plastic waste. Angew. Chem. Int. Ed. 59(36), 15402-15423. (2020).
19 Vollmer, I. et al. Beyond mechanical recycling: Giving new life to plastic waste. Angew. Chem. Int. Ed. 59, 15402-15423(2020).
20 Martin, A. J., Mondelli, C., Jaydev, S. D. & Pérez-Ramírez, J. Catalytic processing of plastic waste on the rise. Chem 7, 1487-1533(2021).
21. Liu, S., Kots, P. A., Vance, B. C., Danielson, A., Vlachos, D. G. Plastic waste to fuels by hydrocracking at mild conditions. Sci. Adv. 7(17), eabf8283 (2021).
22. Jia, X., Qin, C., Friedberger, T., Guan, Z., Huang, Z. Efficient and selective degradation of polyethylenes into liquid fuels and waxes under mild conditions. Sci. Adv. 2(6), e1501591 (2016).
23. Geyer, R., Jambeck, J. R., Law, K. L. Production, use, and fate of all plastics ever made. Sci. Adv. 3(7), e1700782 (2017).
24. Lai, L., Gudiyella, S., Liu, M., Green, W. H. Chemistry of Alkylaromatics Reconsidered. Energy Fuel. 32(4), 5489-5500 (2018).
25. Ratkiewicz, A. Kinetics of the C—C bond beta scission reactions in alkyl radicals. Phys. Chem. Chem. Phys. 13(33), 15037-15046. (2011).
26. Zhang, Y., Cao, C., Li, Y., Yuan, W., Yang, X., Yang, J., Qi, F., Huang, T. P., Lee, Y. Y. Pyrolysis of n-butylbenzene at various pressures: influence of long side-chain structure on alkylbenzene pyrolysis. Energy Fuel. 31(12), 14270-14279 (2017).
27 Fu, D. et al. Elucidating zeolite channel geometry-reaction intermediate relationships for the methanol-to-hydrocarbon process. Angew. Chem. 132, 20199-20205 (2020).
28 DeLuca, M., Janes, C. & Hibbitts, D. Contrasting arene, alkene, diene, and formaldehyde hydrogenation in H-ZSM-5, H—SSZ-13, and H-SAPO-34 frameworks during MTO. ACS Catal. 10, 4593-4607 (2020).
29. llias, S., Bhan, A. Mechanism of the catalytic conversion of methanol to hydrocarbons. ACS Catal. 3(1), 18-31 (2013).
30 Gao, W. et al. Dual active sites on molybdenum/ZSM-5 catalyst for methane dehydroaromatization: Insights from solid-state NMR spectroscopy. Angew. Chem. Int. Ed. 60, 10709-10715 (2021).
31. Li, C., Paris, C., Martínez-Triguero, J., Boronat, M., Moliner, M., Corma, A. Synthesis of reaction-adapted zeolites as methanol-to-olefins catalysts with mimics of reaction intermediates as organic structure-directing agents. Nature Catal. 1(7), 547-554 (2018).
32. Ferri, P., Li, C., Millan, R., Martinez-Triguero, J., Moliner, M., Boronat, M., Corma, A. Impact of Zeolite Framework Composition and Flexibility on Methanol-To-Olefins Selectivity: Confinement or Diffusion?. Angew. Chem. Int. Ed. 59(44), 19708-19715 (2020).
33. Kang, J. H., Alshafei, F. H., Zones, S. I., Davis, M. E. Cage-defining ring: a molecular sieve structural indicator for light olefin product distribution from the methanol-to-olefins reaction. ACS Catal 9(7), 6012-6019 (2019).
34. Meusinger, J., Corma, A. Activation of hydrogen on zeolites: kinetics and mechanism of n-heptane cracking on H-ZSM-5 zeolites under high hydrogen pressure. J. Catal. 152(1), 189-197 (1995).
35. Senger, S., Radom, L. Zeolites as transition-metal-free hydrogenation catalysts: a theoretical mechanistic study. J. Am. Chem. Soc. 122(11), 2613-2620 (2000).

1. A process for the catalytic decomposition of a polyolefin, the process comprising a step of contacting a polyolefin with:
(a) a zeolite, and
(b) an aromatic compound
at a temperature of 150-300° C. and under an inert atmosphere comprising hydrogen;
wherein the zeolite comprises a plurality of Brønsted acid sites and a plurality of pores each having a diameter of 0.45-0.60 nm.

2. The process of claim 1, wherein the polyolefin comprises greater than 80 wt % of PE, PP or a combination thereof.

3. The process of claim 1, wherein the polyolefin is PE, PP or a combination thereof.

4. The process of claim 1, wherein the polyolefin is selected from the group consisting of HDPE, LDPE, LLDPE, HDPP, LDPP, LLDPP and a combination of two or more thereof.

5. The process of claim 1, wherein the polyolefin is selected from the group consisting of HDPE, LDPE, HDPP and a combination of two or more thereof.

6. The process of claim 1, wherein the zeolite is of the MFI framework type.

7. The process of claim 1, wherein the plurality of pores of the zeolite comprise 10-membered ring channels.

8. The process of claim 1, wherein the plurality of pores of the zeolite each have a diameter of 0.48-0.58 nm.

9. The process of claim 1, wherein the zeolite is ZSM-5 having a plurality of BAS.

10. The process of claim 1, wherein the zeolite comprises a SiO₂/Al₂O₃ ratio of 10-200;
or a SiO₂/Al₂O₃ ratio of 20-125.

11. (canceled)

12. The process of claim 1, wherein the zeolite comprises a SiO₂/Al₂O₃ ratio of 30-75.

13. The process of claim 1, wherein the aromatic compound comprises a molecular weight of less than 250 g mol⁻¹; or
a molecular weight of less than 200 g mol⁻¹; or
a molecular weight of less than 175 g mol⁻¹.

14-15. (canceled)

16. The process of claim 1, wherein the aromatic compound comprises a molecular weight of less than 150 g mol⁻¹.

17. The process of claim 1, wherein the aromatic compound is a monocyclic aromatic ring or a bicyclic aromatic ring system, any ring of which is optionally substituted with one or more substituents independently selected from (1-5C)alkyl, (2-5C)alkenyl or (2-5C)alkynyl.

18. The process of claim 1, wherein the aromatic compound is benzene, optionally substituted with one or more substituents independently selected from (1-5C)alkyl, (2-5C)alkenyl or (2-5C)alkynyl.

19. The process of claim 1, wherein the aromatic compound is selected from the group consisting of benzene, toluene, xylene, cumene, mesitylene, 1,2,4,5-tetramethyl benzene and naphthalene.

20. The process of claim 1, wherein the step of contacting the polyolefin with the zeolite and the aromatic compound is conducted at a temperature of 200-300° C.; or
the step of contacting the polyolefin with the zeolite and the aromatic compound is conducted at a temperature of 250-300° C.

21. (canceled)

22. The process of claim 1, wherein the step of contacting the polyolefin with the zeolite and the aromatic compound is conducted at a temperature of 250-280° C.

23. The process of claim 1, wherein the step of contacting the polyolefin with the zeolite and the aromatic compound is conducted in an atmosphere of nitrogen and hydrogen.

24. The process of claim 1, wherein the step of contacting the polyolefin with the zeolite and the aromatic compound is conducted in an atmosphere of hydrogen.
